SEQUENCE LISTING (13 sequences)

SEQ ID NO: 1 - DNA, 23 bp, synthetic construct
  source 1..23: mol_type = other DNA; note = telomere ssDNA
tagggttagggttagggttaggg

SEQ ID NO: 2 - DNA, 23 bp, synthetic construct
  source 1..23: mol_type = other DNA; note = human promoter ssDNA PU22C
ctgagggtgggtagggtgggtaa

SEQ ID NO: 3 - DNA, 28 bp, synthetic construct
  source 1..28: mol_type = other DNA; note = HIV ssDNA LTR-III
gggaggcgtggcctgggcgggactgggg

SEQ ID NO: 4 - DNA, 32 bp, synthetic construct
  source 1..32: mol_type = other DNA; note = wtTel34
gatccccttacccttacccttacccctaggta

SEQ ID NO: 5 - DNA, 31 bp, synthetic construct
  source 1..31: mol_type = other DNA; note = PU30C
agctttacccaccctacccaccctcaggtac

SEQ ID NO: 6 - DNA, 36 bp, synthetic construct
  source 1..36: mol_type = other DNA; note = LTR-C
gatcccccagtcccgcccaggccacgcctccctgca

SEQ ID NO: 7 - DNA, 34 bp, synthetic construct
  source 1..34: mol_type = other DNA; note = repeating unit
ggtacctagggttagggttagggttaggggatcc

SEQ ID NO: 8 - DNA, 1032 bp, synthetic construct
  source 1..1032: mol_type = other DNA; note = fusion sequence with 12 repeating units
ggtacctagggttagggttagggttaggggtaccggtacctagggttagggttagggttaggggtaccggtacctagggttagggttagggttaggggtaccggtacccgccctgctttctcggcatctctgatagcctgagaagaaaccccaactaaatccgctgcttcacctattctccagcgccgggttattttcctcgcttccgggctgtcatcattaaactgtgcaatggcgatagccttcgtcatttcatgaccagcgtttatgcactggttaagtgtttccatgagtttcattctgaacatggtacctagggttagggttagggttaggggtaccggtacctagggttagggttagggttaggggtaccggtacctagggttagggttagggttaggggtaccggtaccgatcggtgggcagtttaccttcatcaaatttgcccattaactcagtttcaatacggtgcagagccagacaggaaggaataatgtcaagccccggccagcaagtgggctttattgcataagtgacatcgtccttttccccaagatagaaaggcaggagagtgtcttctgcatgaatatgaagatctggtacccatccgtgaggtacctagggttagggttagggttaggggtaccggtacctagggttagggttagggttaggggtaccggtacctagggttagggttagggttaggggtaccggtaccataatcagaccgacgatacgagtgggaccgtggtcccagtctgattatcagaccgacgatacgagtgggaccgtggtcccagactaataatcagaccgacgatacgagtgggaccgtggtcccagactaataatcagaccgacgatacgagtgggaccgtggtcccagtctgattatcagaccgacgatacaagtggaacggtacctagggttagggttagggttaggggtaccggtacctagggttagggttagggttaggggtaccggtacctagggttagggttagggttaggggtaccggtacc

SEQ ID NO: 9 - DNA, 33 bp, synthetic construct
  source 1..33: mol_type = other DNA; note = repeating unit
ggtacctgagggtgggtagggtgggtaaagctt

SEQ ID NO: 10 - DNA, 1460 bp, synthetic construct
  source 1..1460: mol_type = other DNA; note = fusion sequence with 20 repeating units
ggtacctgagggtgggtagggtgggtaaagcttggtacctgagggtgggtagggtgggtaaagcttggtacctgagggtgggtagggtgggtaaagcttggtacctgagggtgggtagggtgggtaaagcttatgttcagaatgaaactcatggaaacacttaaccagtgcataaacgctggtcatgaaatgacgaaggctatcgccattgcacagtttaatgatgacagcccggaagcgaggaaaataacccggcgctggagaataggtgaagcagcggatttagttggggtttcttctcaggctatcagagatgccgagaaagcagggcgggtacctgagggtgggtagggtgggtaaagcttggtacctgagggtgggtagggtgggtaaagcttggtacctgagggtgggtagggtgggtaaagcttggtacctgagggtgggtagggtgggtaaagctttcacggatgggaaccagatcttcatattcatgcagaagacactctcctgcctttctatcttggggaaaaggacgatgtcacttatgcaataaagcccacttgctggccggggcttgacattattccttcctgtctggctctgcaccgtattgaaactgagttaatgggcaaatttgatgaaggtaaactgcccaccgatcggtacctgagggtgggtagggtgggtaaagcttggtacctgagggtgggtagggtgggtaaagcttggtacctgagggtgggtagggtgggtaaagcttggtacctgagggtgggtagggtgggtaaagcttgttccacttgtatcgtcggtctgataatcagactgggaccacggtcccactcgtatcgtcggtctgattattagtctgggaccacggtcccactcgtatcgtcggtctgattattagtctgggaccacggtcccactcgtatcgtcggtctgataatcagactgggaccacggtcccactcgtatcgtcggtctgattatggtacctgagggtgggtagggtgggtaaagcttggtacctgagggtgggtagggtgggtaaagcttggtacctgagggtgggtagggtgggtaaagcttggtacctgagggtgggtagggtgggtaaagcttttaatacgatacctgcgtcataattgattatttgacgtggtttgatggcctccacgcacgttgtgatatgtagatgataatcattatcactttacgggtcctttccggtgatccgacaggttacggggcggcgacctcgcgggttttcgctatttatgaaaattttccggtttaaggcgtttccgttcttcttcgtcataggtacctgagggtgggtagggtgggtaaagcttggtacctgagggtgggtagggtgggtaaagcttggtacctgagggtgggtagggtgggtaaagcttggtacctgagggtgggtagggtgggtaaagctt

SEQ ID NO: 11 - DNA, 38 bp, synthetic construct
  source 1..38: mol_type = other DNA; note = repeating unit
ctgcagggaggcgtggcctgggcgggactgggggatcc

SEQ ID NO: 12 - DNA, 1370 bp, synthetic construct
  source 1..1370: mol_type = other DNA; note = fusion sequence with 15 repeating units
ctgcagggaggcgtggcctgggcgggactgggggatccctgcagggaggcgtggcctgggcgggactgggggatccctgcagggaggcgtggcctgggcgggactgggggatccatgttcagaatgaaactcatggaaacacttaaccagtgcataaacgctggtcatgaaatgacgaaggctatcgccattgcacagtttaatgatgacagcccggaagcgaggaaaataacccggcgctggagaataggtgaagcagcggatttagttggggtttcttctcaggctatcagagatgccgagaaagcagggcgctgcagggaggcgtggcctgggcgggactgggggatccctgcagggaggcgtggcctgggcgggactgggggatccctgcagggaggcgtggcctgggcgggactgggggatcctcacggatgggaaccagatcttcatattcatgcagaagacactctcctgcctttctatcttggggaaaaggacgatgtcacttatgcaataaagcccacttgctggccggggcttgacattattccttcctgtctggctctgcaccgtattgaaactgagttaatgggcaaatttgatgaaggtaaactgcccaccgatcctgcagggaggcgtggcctgggcgggactgggggatccctgcagggaggcgtggcctgggcgggactgggggatccctgcagggaggcgtggcctgggcgggactgggggatccgttccacttgtatcgtcggtctgataatcagactgggaccacggtcccactcgtatcgtcggtctgattattagtctgggaccacggtcccactcgtatcgtcggtctgattattagtctgggaccacggtcccactcgtatcgtcggtctgataatcagactgggaccacggtcccactcgtatcgtcggtctgattatctgcagggaggcgtggcctgggcgggactgggggatccctgcagggaggcgtggcctgggcgggactgggggatccctgcagggaggcgtggcctgggcgggactgggggatccttaatacgatacctgcgtcataattgattatttgacgtggtttgatggcctccacgcacgttgtgatatgtagatgataatcattatcactttacgggtcctttccggtgatccgacaggttacggggcggcgacctcgcgggttttcgctatttatgaaaattttccggtttaaggcgtttccgttcttcttcgtcatactgcagggaggcgtggcctgggcgggactgggggatccctgcagggaggcgtggcctgggcgggactgggggatccctgcagggaggcgtggcctgggcgggactgggggatcc

SEQ ID NO: 13 - DNA, 1369 bp, synthetic construct
  source 1..1369: mol_type = other DNA; note = target gene unit PU22
tgcagggaggcgtggcctgggcgggactgggggatccctgcagggaggcgtggcctgggcgggactgggggatccctgcagggaggcgtggcctgggcgggactgggggatccatgttcagaatgaaactcatggaaacacttaaccagtgcataaacgctggtcatgaaatgacgaaggctatcgccattgcacagtttaatgatgacagcccggaagcgaggaaaataacccggcgctggagaataggtgaagcagcggatttagttggggtttcttctcaggctatcagagatgccgagaaagcagggcgctgcagggaggcgtggcctgggcgggactgggggatccctgcagggaggcgtggcctgggcgggactgggggatccctgcagggaggcgtggcctgggcgggactgggggatcctcacggatgggaaccagatcttcatattcatgcagaagacactctcctgcctttctatcttggggaaaaggacgatgtcacttatgcaataaagcccacttgctggccggggcttgacattattccttcctgtctggctctgcaccgtattgaaactgagttaatgggcaaatttgatgaaggtaaactgcccaccgatcctgcagggaggcgtggcctgggcgggactgggggatccctgcagggaggcgtggcctgggcgggactgggggatccctgcagggaggcgtggcctgggcgggactgggggatccgttccacttgtatcgtcggtctgataatcagactgggaccacggtcccactcgtatcgtcggtctgattattagtctgggaccacggtcccactcgtatcgtcggtctgattattagtctgggaccacggtcccactcgtatcgtcggtctgataatcagactgggaccacggtcccactcgtatcgtcggtctgattatctgcagggaggcgtggcctgggcgggactgggggatccctgcagggaggcgtggcctgggcgggactgggggatccctgcagggaggcgtggcctgggcgggactgggggatccttaatacgatacctgcgtcataattgattatttgacgtggtttgatggcctccacgcacgttgtgatatgtagatgataatcattatcactttacgggtcctttccggtgatccgacaggttacggggcggcgacctcgcgggttttcgctatttatgaaaattttccggtttaaggcgtttccgttcttcttcgtcatactgcagggaggcgtggcctgggcgggactgggggatccctgcagggaggcgtggcctgggcgggactgggggatccctgcagggaggcgtggcctgggcgggactgggggatcc

(19) United States
(12) Patent Application Publication
(10) Pub. No.: US 2025/0084447 A1
(43) Pub. Date: Mar. 13, 2025

(54) METHOD FOR PREPARING STABLE ISOTOPE-LABELED ssDNA BY BIOSYNTHESIS WITH ESCHERICHIA COLI

(71) Applicant: EAST CHINA UNIVERSITY OF SCIENCE AND TECHNOLOGY, Shanghai (CN)

(72) Inventors: Shenlin Wang, Shanghai (CN); Mengbing Zou, Shanghai (CN); Changxing Ma, Shanghai (CN)

(21) Appl. No.: 18/649,214

(22) Filed: Apr. 29, 2024

Related U.S. Application Data
(63) Continuation-in-part of application No. PCT/CN2024/079487, filed on Mar. 1, 2024.

(30) Foreign Application Priority Data
Sep. 7, 2023 (CN) 202311152733.2

(51) Int. Cl. C12P 19/34; C12N 15/70

(52) U.S. Cl. CPC C12P 19/34; C12N 15/70

(57) ABSTRACT

Provided is a method for preparing stable isotope-labeled single-stranded DNA (ssDNA) by biosynthesis with E. coli, and the ¹⁵NH₄Cl or ¹³C-Glcose is used as the only nitrogen or carbon source, which may significantly reduce costs. In the method of the present disclosure, the target sequence of ssDNA is tandemly duplicated on a high-copy vector, a site for a first restriction endonuclease and a site for a second restriction endonuclease are added to the 5′ and 3′ ends of the target sequence, respectively, and the recombinant vector is digested to obtain an asymmetric double-stranded DNA structure, which is then isolated by denaturation to obtain two ssDNAs of unequal lengths, including ¹⁵N- or ¹³C-labeled target ssDNA. The method of the present disclosure is able to effectively increase the yield of ssDNA, thereby improving the efficiency of in vitro synthesis of isotope labeled ssDNA.

### CROSS-REFERENCE TO RELATED APPLICATION(S)

The present application is a continuation of the International Patent Application No. PCT/CN2024/079487, filed on Mar. 1, 2024, which claims the priority of Chinese Patent Application No. CN202311152733.2 filed with the China National Intellectual Property Administration on Sep. 7, 2023, the disclosure of each of which is incorporated by reference herein in its entirety as part of the present application.

### REFERENCE TO SEQUENCE LISTING

A computer readable XML file entitled “GWP20230907126_seglist.xml”, which was created on Oct. 24, 2023, with a file size of 19,962 bytes as measured in the Microsoft Windows operating system, contains the sequence listing for this application, is filed electronically with this application, and is hereby incorporated by reference in its entirety.

### TECHNICAL FIELD

The present disclosure belongs to the technical field of DNA synthesis, and specifically relates to a method for preparing stable isotope-labeled single-stranded DNA by biosynthesis with E. coli and enzymatic digestion.

### BACKGROUND

Most DNA has a double helix structure, which is a double-stranded form but changes to a single-stranded state as soon as it is treated with heat or bases. Single-stranded DNA (ssDNA) refers to DNA that exists in single strand state. ssDNA is different from double-stranded DNA (dsDNA) in terms of molecular hydrodynamic properties, absorption spectroscopy, base-reaction properties, three-dimensional (3D) structure, and function. Notably, ssDNA can fold to form higher-order structures that play important roles in life processes, such as the G-quadruplex (G4). Stable isotope labeling of ssDNA can facilitate 3D structure determination of ssDNA by Nuclear Magnetic Resonance (NMR).

The current commercial method to obtain stable isotope-labeled ssDNA is the solid-phase synthesis method, which has an expensive synthesis cost. The synthesis of 1 mg of 24 nt stable isotope-labeled ssDNA costs about 330,000 RMB. Moreover, for DNA sequence with high GC content, the preparation process often encounter problems such as base pair mismatch or hindering of polymerase action by secondary structure. Therefore, it is urgently needed in the field to develop a universal method for preparing isotope-labeled ssDNA.

### SUMMARY

Embodiments of the present disclosure provide a method for preparing stable isotope-labeled ssDNA by biosynthesis with Escherichia coli (E. coli). The method of the present disclosure is a universal method for labeling ssDNA with ¹⁵N and/or ¹³C, which is able to effectively increase the efficiency of the in vitro synthesis of ¹⁵N and/or ¹³C-labeled ssDNA and to reduce the synthesis cost.

The present disclosure provides a method for preparing stable isotope-labeled ssDNA by biosynthesis with E. coli and enzymatic digestion, including the following steps:
1) Adding a site of a first restriction endonuclease and a site of a second restriction endonuclease to 5′ and 3′ ends of a target sequence, respectively, to obtain a repeating unit; where
the first restriction endonuclease and the second restriction endonuclease are different;
2) ligating the repeating unit obtained in step 1) in tandem to obtain a fusion sequence;
3) inserting the fusion sequence in step 2) into a high-copy vector to obtain a recombinant vector;
4) introducing the recombinant vector in step 3) into a host strain to obtain a recombinant E. coli strain;
5) extracting and purifying the recombinant vector from the recombinant E. coli strain after culture of the recombinant E. coli strain in step 4); where the culture is conducted in a medium with ¹⁵NH₄Cl as a sole nitrogen source and/or ¹³C-glucose as a sole carbon source;
6) digesting the recombinant vector obtained in step 5) using the first restriction endonuclease and the second restriction endonuclease to obtain an asymmetric dsDNA, and isolating two ssDNAs of unequal lengths, with one of the two ssDNAs as a target sequence; and
7) recovering the target sequence in the two ssDNAs in step 6) to obtain an isotope-labeled target ssDNA.

In some embodiments, in step 1), the first restriction endonuclease is KpnI and the second restriction endonuclease is BamHI;
alternatively, the first restriction endonuclease is KpnI-HF® and the second restriction endonuclease is BamHI-HF®;
alternatively, the first restriction endonuclease is Pst I-HF® and the second restriction endonuclease is Hind III-HF®;
alternatively, the first restriction endonuclease is Kpn I-HF® and the second restriction endonuclease is Hind III-HF®.

In some embodiments, in step 6), the isolating is isolating by urea-polyacrylamide gel electrophoresis.

In some embodiments, a short one of the two ssDNAs of unequal lengths in step 4) is a target sequence.

In some embodiments, in step 2), the ligating is conducted by ligating every 3 to 4 repeating units in tandem as a large repeating unit, and then ligating the large repeating unit in tandem by a linker sequence.

In some embodiments, in step 2), the fusion sequence has a length of not more than 2000 nt.

In some embodiments, in step 3), the high-copy vector includes pUC57.

In some embodiments, in step 1), the target sequence includes telomere ssDNA, human cellular homolog of avian myelocytomatosis virus oncogene (c-Myc) promoter ssDNA or human immunodeficiency virus (HIV) ssDNA; the telomere ssDNA has the nucleotide sequence set forth in SEQ ID NO: 1; the human c-Myc promoter ssDNA has the nucleotide sequence set forth in SEQ ID NO: 2; the HIV ssDNA has the nucleotide sequence set forth in SEQ ID NO: 3.

In some embodiments, the ligating in step 2) is conducted by ligating 12 repeating units in tandem to obtain the fusion sequence when the target sequence is telomere ssDNA;
the ligating is conducted by ligating 20 repeating units are ligated in tandem to obtain the fusion sequence when the target sequence is human c-Myc promoter ssDNA; and
the ligating is conducted by ligating 15 repeating units in tandem to obtain the fusion sequence when the target sequence is HIV ssDNA.

In some embodiments, ¹⁵NH₄Cl in the medium in step 5) has a concentration of at least 1 g/L; the ¹³C-glucose in the medium has a concentration of at least 4 g/L.

In some embodiments, step 7) further includes recovering another ssDNA in addition to the target sequence.

The present disclosure provides a method for preparing stable isotope-labeled ssDNA by biosynthesis with E. coli. The target sequence of the ssDNA is ligated in a tandemly duplicated way on the high-copy vector, a site for the first restriction endonuclease and a site for the second restriction endonuclease are added at the 5′ and 3′ ends of the target sequence, respectively, the recombinant vector is digested, an asymmetric dsDNA structure are obtained, and then two ssDNAs of unequal lengths are isolated, including ¹⁵N and/or ¹³C-labeled target ssDNAs. The method of the present disclosure is able to effectively increase the yield of ssDNA, and thus improve the efficiency of in vitro synthesis of ssDNA. Moreover, the medium used for culturing the host strain of the present disclosure has ¹⁵NH₄Cl as the sole nitrogen source and/or ¹³C-glucose as the sole carbon source, which may effectively reduce the cost of stable isotope-labeled DNA synthesis.

The present summary is provided only by way of example and not limitation. Other aspects of the present invention will be appreciated in view of the entirety of the present disclosure, including the entire text, claims, and accompanying figures.

### BRIEF DESCRIPTION OF THE DRAWINGS

In order to more clearly illustrate the technical solutions in the examples or prior art of the present disclosure, the accompanying drawings to be used in the examples will be briefly introduced below.

FIG. 1 is a schematic diagram of ¹⁵N labeled telomere ssDNA in Example 1, where A shows the formation of the plasmid pUC57-wtTel23c; B shows the propagation of the constructed plasmid by transforming it into DH5a Escherichia coli cells; C shows the propagation of the DH5a E. coli cells transformed with pUC57-wtTel23c grown in M9 medium, with ¹⁵N-labeled NH₄Cl as the sole nitrogen source to produce ¹⁵N-labeled pUC57-wtTel23c plasmid; D shows the purified ¹⁵N-labeled pUC57-wtTel23c plasmid treated with restriction endonuclease KpnI and BamHI; F shows the digested product obtained as an asymmetric dsDNA; G shows the further isolation and purification of the asymmetric dsDNA by denaturing urea polyacrylamide gel electrophoresis (PAGE); and H shows the recovery and electroelution of the target ssDNA to obtain the target ¹⁵N-labeled ssDNA;

FIGS. 2A and 2B show the identification of ¹⁵N-labeled wtTel23c, where FIG. 2A shows the mass spectrometry analysis result of ¹⁵N-labeling yield of ¹⁵N-labeled wtTel23c, reference is made to the reference ssDNA wtTel23c synthesized by solid-phase synthesis, NA-wtTel23c is the isotopically labeled wtTel23c with natural abundance prepared by biosynthesis via the method of the present disclosure, and ¹⁵N-wtTel23c is ¹⁵N-labeled wtTel23; FIG. 2B shows the proton region of the imino group in the 1D ¹H-NMR spectrum of G4 formed from wtTel23c and the reference, respectively; FIGS. 2C and 2D show the imino region in the ¹H-¹⁵N 2D sfHMQC (¹H-¹⁵N band-selective optimized flip-angle short-transient (SOFAST) heteronuclear multiple quantum coherence (HMQC)) of G4 formed from wtTel23c and the reference, respectively, the reference being ssDNA with the same sequence as that in the solid-phase synthesis;

FIG. 3 shows the results of the comparison of the ¹H-¹⁵N 2D sfHMQC of the wtTel23c-TMPyP4 complex in the cell-like environment and the K⁺ dilute solution, in which the dashed line spectrum indicates the signal of the wtTel23c-TMPyP4 complex in the K⁺ dilute solution; the solid line spectrum indicates the signal of the wtTel23c-TMPyP4 complex in the cell-like environment;

FIG. 4 shows a schematic diagram of ¹⁵N labeling of human c-Myc promoter c-Myc ssDNA in Example 3, where the blank part of the plasmid indicates the sequence of the pUC57 vector; the diagonals indicate the BsmB I cleavage site; the grid indicates the tandemly duplicated target gene (PU22)₂₀, and the partial enlarged detail indicates a target gene unit PU22 (SEQ ID NO: 13, the difference from PU22C is that there is one less C base at the 5′ end), where ellipses indicate tandem repeats of the target gene, the sequence pointed by the hollow arrowhead indicates the double-stranded DNA sequence of pUC57-PU22C obtained by double digestion with Hind III-HF and Kpn I-HF of unequal lengths; and two ssDNAs of unequal lengths, PU22C and PU30C, are obtained by denaturing PAGE and purification;

FIGS. 5A-5C shows the results of gel detection and digestion of the plasmid in Example 5, where FIG. 5A shows the purified plasmid pUC57-PU22C detected by 1% agarose gel electrophoresis; FIG. 5B shows the detection results of stepwise double digestion of the plasmid by 8% acrylamide gel electrophoresis, where lane Hind III-HF indicates the plasmid subjected to Hind III-HF digestion, and lane Kpn I-HF indicates the plasmid subjected to Hind III-HF followed by Kpn I-HF digestion; FIG. 5C shows ssDNA (PU22C) obtained by 12% acrylamide gel electrophoresis, where lane Reference indicates solid-phase synthesized ssDNA with the same sequence as PU22C;

FIGS. 6A-6D show the ¹H-¹⁵N 2D sfHMQC NMR spectra of the mutual titration of CX5461 with PU22C, where the solid line spectrum indicates PU22C in the free state and the dashed line spectrum indicates PU22C in the bound state, and the ratios labeled in the figures are the titration ratio of PU22C to CX5461;

FIGS. 7A-7D show the ²J_{HN}-¹H-¹⁵N HSQC NMR spectra of the mutual titration of CX5461 with PU22C, where the solid line spectrum indicates PU22C in the free state and the dashed line spectrum indicates PU22C in the bound state, and the ratios labeled in the figures are the titration ratios of PU22C to CX5461;

FIG. 8 shows a schematic diagram of ¹⁵N labeling of HIV ssDNA in Example 6, where the blank portion of the plasmid indicates the pUC57 vector sequence, and the diagonals indicate the BsmB I enzymatic site; the grid indicates the tandemly duplicated target gene (long terminal repeat (LTR)-III)₁₆, the partially enlarged view indicates a target gene unit LTR-III, ellipsis denotes the tandemly repeated target genes, and the sequence pointed by the hollow arrowhead indicates the double-stranded DNA sequences of unequal lengths obtained by double digestion of pUC57-LTR-III with Pst I-HF and Hind III-HF; two ssDNAs of unequal lengths, LTR-III and LTR-C, are obtained by denaturing urea PAGE purification;

FIG. 9A-9D show the fingerprints of the interaction of LTR-III G4 with the ligand Braco-19 from the acquisition of ¹H-¹⁵N 2D sfHMQC spectra, in which the molar ratio of G4 to ligand interaction (G4:Braco-19) is shown in the upper left corner of the figures;

FIGS. 10A-10C show the effect of complex cellular environment on the conformation of LTR-III G4, in which the concentration of cell lysate is shown in the upper left corner of the figures; the gray spectrum indicates the spectral information of LTR-III G4 without cell lysate; the black spectral figures (FIGS. 10A-10C) indicate the spectral information of LTR-III G4 obtained under the conditions of different concentrations of cell lysate;

FIGS. 11A-11C show the interaction of LTR-III G4 with the ligand Braco-19 in a complex cellular environment, where the gray background represents the spectral signal of LTR-III G4 in free state at a cellular concentration of 50 mg/mL; and the red spectra represents the spectral information of the complexes with different titration ratios under the conditions of cellular extracts; and

FIG. 12 shows the ¹H-¹³C 2D HSQC spectra characterizing the C—H signals of various types of bases.

### DETAILED DESCRIPTION OF THE EMBODIMENTS

The present disclosure provides a method for preparing stable isotope-labeled ssDNA by biosynthesis with E. coli, including the following steps:
1) adding a site of a first restriction endonuclease and a site of a second restriction endonuclease to 5′ and 3′ ends of a target sequence, respectively, to obtain a repeating unit; where
the first restriction endonuclease and the second restriction endonuclease are different;
2) connecting the repeating unit in step 1) in tandem to obtain a fusion sequence;
3) inserting the fusion sequence in step 2) into a high-copy vector to obtain a recombinant vector;
4) introducing the recombinant vector in step 3) into host strain to obtain a recombinant E. coli strain;
5) extracting and purifying the recombinant vector from the recombinant E. coli strain after culture of the recombinant E. coli strain in step 4); where the culture is conducted in a medium with ¹⁵NH₄Cl as a sole nitrogen source and/or ¹³C-glucose as a sole carbon source;
6) digesting the recombinant vector in step 5) using the first restriction endonuclease and the second restriction endonuclease to obtain an asymmetric dsDNA structure, and isolating two ssDNAs of unequal lengths, with one of the two ssDNAs as a target sequence; and
7) recovering the target sequence in the two ssDNAs in step 6) to obtain a stable isotope-labeled target ssDNA.

In the present disclosure, the site for the first restriction endonuclease and the site for the second restriction endonuclease are firstly added to the 5′ and 3′ ends of the target sequence, respectively, to obtain the repeating unit.

In the present disclosure, the length of the target sequence is preferably less than 100 nt.

In the present disclosure, the target sequence preferably includes telomere ssDNA, human c-Myc promoter ssDNA or HIV ssDNA; the nucleotide sequence of the telomere ssDNA is preferably set forth in SEQ ID NO: 1, specifically: 5′-tagggttagggttagggttaggg-3′, designated as wtTel23; the nucleotide sequence of the human c-Myc promoter c-Myc ssDNA is preferably set forth in SEQ ID NO: 2, specifically: 5′-ctgagggtgggtagggtgggtaa-3′, designated as PU22C; the nucleotide sequence of the HIV ssDNA is preferably set forth in SEQ ID NO: 3, specifically: 5′-gggaggcgtggcctgggcgggactgggg-3′, designated as LTR-III.

In the present disclosure, the first restriction endonuclease is preferably KpnI and the second restriction endonuclease is preferably BamHI; or, the first restriction endonuclease is preferably KpnI-HF and the second restriction endonuclease is preferably BamHI-HIF; or, the first restriction endonuclease is preferably Pst I-HF and the second restriction endonuclease is preferably Hind III-HF; or, the first restriction endonuclease is preferably Kpn I-HF and the second restriction endonuclease is preferably Hind III-HF. The above combinations of restriction endonucleases are highly efficient and ensure that the shorter one of the two ssDNAs of unequal lengths obtained by subsequent programs is a target sequence.

In the present disclosure, when the target sequence is telomere ssDNA, the first restriction endonuclease is preferably KpnI and the second restriction endonuclease is preferably BamHI.

In the present disclosure, when the target sequence is human c-Myc promoter ssDNA, the first restriction endonuclease is preferably Kpn I-HF and the second restriction endonuclease is preferably Hind III-HF.

In the present disclosure, when the target sequence is HIV ssDNA, the first restriction endonuclease is preferably Pst I-HF and the second restriction endonuclease is preferably Hind III-HF.

After the repeating units are obtained, the repeating units in the present disclosure are ligated in tandem to obtain the fusion sequence.

In the present disclosure, every 3 to 4 repeating units are ligated in tandem as a large repeating unit, and the large repeating unit is then ligated in tandem through a linker sequence; the linker sequence preferably does not contain repeat sequences, and has a length greater than the target sequence, so as to facilitate the subsequent isolation. In the present disclosure, the length of the linker sequence preferably ranges from 50 to 200 nt; the advantage of ligating in tandem through the linker sequence is that it is less difficult to synthesize the plasmid. In the present disclosure, the length of the fusion sequence is preferably not more than 2 k, more preferably 1.3 to 2 k, which range not only meets the requirements for constructing recombinant vectors, but also increases the number of repetitions of the target sequences in order to improve their yield.

In the present disclosure, when the target sequence is telomere ssDNA, the fusion sequence is preferably obtained from 12 repeating units in tandem; in some other embodiments, every 3 repeating units in tandem constitute a large repeating unit.

In the present disclosure, when the target sequence is a human c-Myc promoter ssDNA, the fusion sequence is preferably obtained from 20 repeating units in tandem; in some other embodiments, every 4 repeating units in tandem constitute a large repeating unit.

In the present disclosure, when the target sequence is HIV ssDNA, the fusion sequence is preferably obtained by tandemly fusing 15 repeating units; in some other embodiments, every 3 repeating units are tandemly fused as a large repeating unit.

After the fusion sequence is obtained, the fusion sequence in the present disclosure is inserted into a high-copy vector to obtain a recombinant vector.

In the present disclosure, the high-copy vector preferably includes pUC57, purchased from Genescript Biotech Corporation (Nanjing, China).

In the present disclosure, when the target sequence is telomere ssDNA, the fusion sequence is preferably ligated to the pUC57 vector at a BsmBI restriction site.

In the present disclosure, when the target sequence is a human c-Myc promoter ssDNA, the fusion sequence is preferably ligated to the pUC57 vector at an EcoRV restriction site.

In the present disclosure, when the target sequence is HIV ssDNA, the fusion sequence is preferably ligated to the pUC57 vector at a BsmBI restriction site.

After the recombinant vector is obtained, the recombinant vector in the present disclosure is introduced into host strain to obtain the recombinant E. coli strain.

In the present disclosure, the host strain preferably include E. coli; the E. coli preferably includes DH5α E. coli, and the probability of mutation for DNA replication within a DH5α E. coli cell is very low, which is about 10⁻¹⁰, four orders of magnitude lower than that of a high fidelity PCR polymerase.

After the recombinant E. coli strain are obtained, the recombinant bacteria in the present disclosure are cultured and the recombinant vector are extracted and purified therefrom; a medium with ¹⁵NH₄Cl as the sole source of nitrogen and/or ¹³C-glucose as the sole source of carbon are used in the culture.

In the present disclosure, the medium preferably includes M9 medium containing ¹⁵NH₄Cl and/or ¹³C-glucose, and it is advantageous to use ¹⁵NH₄Cl and/or ¹³C-glucose as the raw material for biosynthesis with E. coli, which is of low cost; the ¹⁵NH₄Cl in the medium preferably has a concentration of at least 1 g/L, more preferably 1 g/L; the ¹³C-glucose in the medium preferably has a concentration of at least 4 g/L, more preferably 4 g/L, which is the minimum amount to ensure that the culture medium has sufficient nitrogen and carbon sources at the same time, and, may save costs.

After the extracted and purified recombinant vector is obtained, a first restriction endonuclease and a second restriction endonuclease is used in the present disclosure to digest the extracted and purified recombinant vector, to obtain an asymmetric double-stranded DNA structure, and two ssDNAs of unequal lengths are isolated and obtained, with one of the two ssDNAs being the target sequence.

In the present disclosure, isolating and obtaining two ssDNAs of unequal lengths preferably includes isolating and obtaining two ssDNAs of unequal lengths by denaturing urea PAGE, which has the advantage of simple operation and low cost.

In the present disclosure, the denaturing urea PAGE is preferably a denaturing urea PAGE at a volume concentration of 12%, which indicates the volume fraction of acrylamide in the polyacrylamide gel; the denaturing urea PAGE preferably includes the following components in 500 mL: 10×TBE 50 mL, 40% ACRY (19:1) 150 mL, 210 g of urea and the balance of water.

After two ssDNAs are obtained, the target sequences in the two ssDNAs of the present disclosure are recovered to obtain a stable isotope-labeled target ssDNA.

If the complementary strand of the target sequence is also of value for research, the present disclosure preferably also includes the recovery of another ssDNA in addition to the target sequence, i.e., the recovery of the complementary strand of the target sequence, so that two different ¹⁵N- and/or ¹³C-labeled ssDNAs can be obtained in one preparation.

The method of the present disclosure for preparing ¹⁵N-labeled ssDNA is a biosynthesis method with E. coli, which is capable of synthesizing ¹⁵N-labeled ssDNA with high labeling efficiency (as high as 96%), low synthesis cost (90% lower cost compared with commercial products), high yield (1L M9 medium may produce 8.1 g ¹⁵N-labeled plasmid), low mutation rate (mutation rate is 10⁻¹⁰, which is reduced by two orders of magnitude with respect to that of high-fidelity PCR polymerization reaction) and other advantages. The method is suitable for the synthesis of common ssDNA and ssDNA sequences rich in repetitive sequences.

In one embodiment of the present disclosure, when the target sequence is telomere ssDNA, the fusion sequence is preferably obtained by tandem ligation of 12 repeating units; every 3 repeating units in tandem are a large repeating unit; the first restriction endonuclease is preferably KpnI and the second restriction endonuclease is preferably BamHI; the high-copy vector is pUC57; the fusion sequence is ligated to the pUC57 vector at the BsmBI restriction site; two ssDNAs of unequal lengths are the short target sequence wtTel23c and the long complementary wtTel34; the nucleotide sequence of the wtTel34 is set forth in SEQ ID NO: 4, specifically: 5′-gatccccttacccttacccttacccctaggta-3′. The ¹⁵N-labeled telomere ssDNA prepared by the method of the present disclosure may drastically reduce the acquisition time of ¹H-¹⁵N 2D sfHMQC spectra, greatly improve the efficiency of the spectrum acquisition, and lay the foundation for characterizing the structure of G4 and its interaction with ligands in a cellular environment.

In one embodiment of the present disclosure, when the target sequence is a human c-Myc promoter ssDNA, the fusion sequence is preferably obtained by tandem ligation of 20 repeating units; every 4 repeating units in tandem are a large repeating unit; the first restriction endonuclease is preferably Kpn I-HF and the second restriction endonuclease is preferably Hind III-HF; the high-copy vector is pUC57; the fusion sequence is ligated to the pUC57 vector at the EcoRV restriction site ligated to the pUC57 vector; two ssDNAs of unequal lengths are the short target sequence PU22C and the long PU30C sequence; the nucleotide sequence of the PU30C is set forth in SEQ ID NO: 5, specifically: 5′-agctttacccaccctacccaccctcaggtac-3′. Compared to the human c-Myc promoter sequence having natural abundance, the ¹⁵N uniformly labeled human c-Myc promoter ssDNA sequence prepared by the method of the present disclosure has the following two advantages: (1) it may improve the sensitivity of NMR detection and substantially increase the efficiency of the acquisition of the NMR ¹H-¹⁵N two-dimensional NMR spectra. Because 99.63% of the nitrogen atoms with natural abundance is in the form of ¹⁴N with a spin of 1 and a quadrupole moment of 2.044 fm². The natural abundance of ¹⁵N is 0.364%, with a spin of ½. Although ¹⁴N has high abundance and spin, it has quadrupole moment, which leads to fast relaxation and wide spectra, and it is not suitable for two-dimension (2D) 1 NMR spectrum acquisition. The full width half maximum (FWHM) of ¹⁵N in spectra is narrow, which is more conducive to analyze the precise structural information of biological macromolecules. Therefore, the usually acquired nitrogen spectra of biomolecules are specifically referred to ¹⁵N NMR spectra. Therefore, increasing the abundance of ¹⁵N may improve the sensitivity of ¹⁵N signal, which in turn improves the acquisition efficiency of the spectra. (2)¹⁵N homogeneously labeled human c-Myc promoter sequences may be used to study the G4 topology of human c-Myc promoters and their interactions with ligands in complex cellular environments. In the cellular environment, when the high abundance ¹⁵N is added to uniformly label human c-Myc promoter G4, all other background molecules are present at the natural abundance (0.364%) of ¹⁵N except for the target human c-Myc promoter G4 which is ¹⁵N labeled, so there is little background interference in its NMR spectroscopy signals, such that the target molecule ¹⁵N signals can be directly collected. Therefore, when ¹⁵N-G4 signal acquisition, such as 2D ¹H-¹⁵N HSQC or 2D HMQC spectrum acquisition, is performed in cellular in situ, the background signal is negligible since the background signal is small.

In one embodiment of the present disclosure, when the target sequence is HIV ssDNA, the fusion sequence is preferably obtained by tandem ligation of 15 repeating units; every 3 repeating units in tandem are a large repeating unit; the first restriction endonuclease is preferably Pst I-HF and the second restriction endonuclease is preferably Hind III-HF; the high-copy vector is pUC57; the fusion sequence is ligated to the pUC57 vector at a BsmBI restriction site; the two ssDNAs of unequal lengths are the short target sequence, LTR-III, and the long by-product, LTR-C, respectively; the nucleotide sequence of the LTR-C is set forth in SEQ ID NO: 6, specifically 5′-gatcccccagtcccgcccaggccacgcctccctgca-3′; the nucleotide sequence of the LTR-III is set forth in SEQ ID NO: 3, specifically 5′-gggaggcgtggcctgggcgggactgggg-3′. The ¹⁵N homogeneously labeled HIV ssDNA sequence LTR-III prepared by the method of the present disclosure improves the sensitivity of NMR signal acquisition and may be used for conventional ¹⁵N-filtered NMR spectrum acquisition. In addition, the ¹⁵N homogeneously labeled HIV ssDNA prepared by the method of the present disclosure may also be used for studies of the structure of HIV LTR-III G4 and its interactions with ligands in complex environments of cellular extracts by NMR. It provides a new platform for screening of drugs targeting HIV LTR-III G4 in complex cellular environments.

In the present disclosure, ssDNAs, including wtTel34, PU30C, and LTR-C, are rich in base C, which facilitates the folding to form functional i-motif structures, and may also be used for NMR studies of i-motif structures and their interactions with ligands.

In order to further illustrate the present disclosure, a method for preparing stable isotope-labeled ssDNA by biosynthesis with E. coli provided by the present disclosure is described in detail below in conjunction with the accompanying drawings and examples, but they are not to be construed as limiting the protection scope of the present disclosure.

In the examples of the present disclosure, the high-copy vector pUC57 was purchased from Nanjing Kingsley, and the fusion sequence was synthesized by Genescript Biotechnology Co. (Nanjing, China).

### Example 1 Preparation of ¹⁵N-Labeled Telomere ssDNA

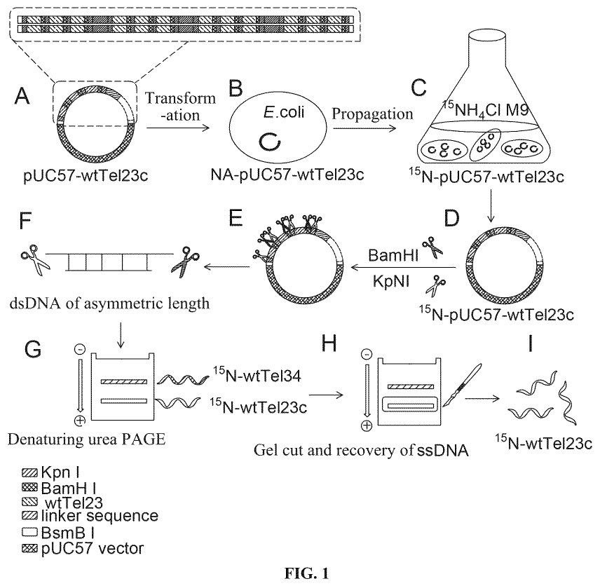

The experimental principle of plasmid design is shown in FIG. 1, and the target sequence of this example was a telomere ssDNA, 5′-tagggttagggttagggttaggg-3′(SEQ ID NO: 1), which was named as wtTel23.

The overall design of the plasmid was used for replication by in vivo cloning to obtain ¹⁵N-labeled ssDNA. To increase the yield of the target DNA, two different restriction sites, Kpn I and BamH I, were added to the 5′ and 3′ ends of the target sequence (bolded font) respectively, to obtain a repeating unit, and the repeating unit nucleotide sequence is set forth in SEQ ID NO: 7, specifically: 5′-ggtacctagggttagggttagggttaggggatcc-3′. The repeating unit was tandemly ligated 12 times to obtain the fusion sequence (the nucleotide sequence is set forth in SEQ ID NO: 8, specifically:

5′-GGTACCTAGGGTTAGGGTTAGGGTTAGGGGTACCGGTACCTAGGGTTAGGGTT
AGGGTTAGGGGTACCGGTACCTAGGGTTAGGGTTAGGGTTAGGGGTACCGGTACC
cgccctgctttctcggcatctctgatagcctgagaagaaaccccaactaaatccgctgcttcacctattctccagcgccgggttattttcct
cgcttccgggctgtcatcattaaactgtgcaatggcgatagccttcgtcatttcatgaccagcgtttatgcactggttaagtgtttccatga
gtttcattctgaacat(Linker)GGTACCTAGGGTTAGGGTTAGGGTTAGGGGTACCGGTACCTAGGGT
TAGGGTTAGGGTTAGGGGTACCGGTACCTAGGGTTAGGGTTAGGGTTAGGGGTAC
CGGTACCgatcggtgggcagtttaccttcatcaaatttgcccattaactcagtttcaatacggtgcagagccagacaggaaggaataat
gtcaagccccggccagcaagtgggctttattgcataagtgacatcgtccttttccccaagatagaaaggcaggagagtgtcttctgcatgaat
atgaagatctGGTACCcatccgtga(Linker)GGTACCTAGGGTTAGGGTTAGGGTTAGGGGTAC
CGGTACCTAGGGTTAGGGTTAGGGTTAGGGGTACCGGTACCTAGGGTTAGGGTTA
GGGTTAGGGGTACCGGTACCataatcagaccgacgatacgagtgggaccgtggtcccagtctgattatcagaccgacg
atacgagtgggaccgtggtcccagactaataatcagaccgacgatacgagtgggaccgtggtcccagactaataatcagaccgacgatac
gagtcggaccgtggtcccagtctgattatcagaccgacgatacaagtggaac(Linker)GGTACCTAGGGTTAGGG
TTAGGGTTAGGGGTACCGGTACCTAGGGTTAGGGTTAGGGTTAGGGGTACCGGTA
CCTAGGGTTAGGGTTAGGGTTAGGGGTACCGGTACC-3′.

Every three repeating units were ligated to a linker that has a random sequences (yellow) to facilitate plasmid construction, and the fusion sequences were ligated in tandem at the BsmBI restriction site to the pUC57 vector to form plasmid pUC57-wtTel23c (see A in FIG. 1). The constructed plasmid was transformed into DH5α E. coli cells for propagation (see B in FIG. 1). DH5α E. coli cells transformed with pUC57-wtTel23c were grown in M9 medium with ¹⁵N-labeled NH₄Cl as the sole nitrogen source to produce the ¹⁵N-labeled pUC57-wtTel23c plasmid (see C in FIG. 1). The purified ¹⁵N-tagged pUC57-wtTel23c plasmid was treated with restriction endonucleases KpnI and BamHI (see D and E in FIG. 1). Asymmetric double-stranded DNA structures were generated by BamH I and Kpn I double-digestion, and two ssDNAs of unequal lengths, wtTel23c: 5′-tagggttagggttagggttaggg-3′ (SEQ ID NO: 1); wtTel34: 5′-gatccccttacccttacccttacccctaggta-3′ (SEQ ID NO: 4), were obtained by isolation using denaturing urea PAGE. The digestion product was obtained as asymmetric dsDNA (see F in FIG. 1). The asymmetric dsDNA was further isolated and purified by denaturing urea PAGE (45 cm×35 cm) (see G in FIG. 1). The target ssDNA was recovered and electroeluted to obtain the target ¹⁵N-labeled ssDNA (see H in FIG. 1).

### Protocols:

### 1. Amplification of Stable Isotope-Labeled Plasmids Using Labeled M9 Medium

The monoclonal colonies were selected and cultured in 5 mL of TB medium (37° C.). After 5 h, 5 mL of the bacterial solution was transferred to 50 mL of TB medium. After 3 h, the bacteria were collected by centrifugation, and the bacteria were transferred to 150 mL of M9 medium containing ¹⁵NH₄Cl at a concentration of 1 g/L for overnight incubation. 50 mL of overnight culture medium was taken and added to 400 mL of fresh M9 medium containing ¹⁵NH₄Cl. Chloramphenicol at a final concentration of 11.5 mg/mL was added when OD=1.0, and the incubation is continued at 37° C. for 9 h. Centrifugation was performed to collect the E. coli cells, which were then washed with Sodium Chloride-Tris-EDTA (STE) buffer for further purification.

### 2. Plasmid Mass Extraction and Purification

Since the mass extraction plasmid kits available on the market could not meet the experimental requirements, the buffer solution required for mass extraction of plasmids was prepared according to the recipe in the Molecular Cloning Book (3rd edition).

The buffer solutions used in the following experimental steps used water as solvent and are formulated as shown below in an amount suitable for 2 L of M9 cultured E. coli cells.
STE buffer: 10 mM Tris-HCl pH 8.0, 100 mM NaCl, 1 mM EDTA pH 8.0, autoclaved, pre-cooled at 4° C. before use;
S1 solution: 25 mM Tris-HCl pH 8.0, 50 mM glucose, 10 mM EDTA pH 8.0, autoclaved and used at room temperature;
S2: 0.2 M NaOH, 1% m/V SDS, fresh made for each time, and used at room temperature;
S3 solution: 3 M KAc, 2 M HAc, pre-cooled at 4° C. before use;
Isopropyl alcohol: used at room temperature;
Aqueous solution of ethanol at 70% by volume (70% ethanol): pre-cooled at 4° C. before use;
4 M NaCl: autoclaved;
40% PEG 6000: autoclaved;
TE 10/0.1: 10 mM Tris-HCl pH 8.0, 0.1 mM EDTA, autoclaved;
TE 10/50: 10 mM Tris-HCl pH 8.0, 50 mM EDTA, autoclaved;
3 M NaAc: pH 5.2.

### 2.1 Plasmid Extraction

(1) The E. coli cells were suspended in 60 mL of pre-cooled S1 solution, 120 mL of S2 was slowly added, gently inverted to mix well, and left to stand at room temperature for 3-5 min, where the resulting mixture was homogeneous and transparent, with high viscosity, and being stringy; then 210 mL of pre-cooled S3 solution was added, and shaken in the same direction until the liquid phase was not stratified.
(2) Incubation was carried out on ice for 10 min.
(3) Centrifugation was performed at 7500 rpm for 20 min and bulk precipitate was removed by filtration through a membrane.
(4) 0.52 volume of isopropanol was added and left to stand for 15 min at room temperature.
(5) The precipitate was collected by centrifugation and washed by adding an appropriate amount of pre-cooled 75% ethanol.
(6) The ethanol solution was centrifuged and the residual ethanol was removed by volatilization.
(7) An appropriate volume of TE10/50 was added to dissolve the precipitate.
(8) 160 μL of RNaseA (25 mg/mL) was added and digested at 37° C. overnight.
(9) Hydrolysis of RNA was detected by 1% agarose gel electrophoresis.

### 2.2. Plasmid Purification

(1) 1/5 volume of 4 M NaCl, 2/5 volume of 40% PEG6000 were added and left to stand at 37° C. for 5 min.
(2) Incubation was conducted for 1 h on ice.
(3) Centrifugation was performed at 13000 rpm for 30 min, and the precipitate was collected and washed with 70% ethanol.
(4) The ethanol was removed, the residual ethanol was removed by volatilization, and the precipitate was dissolved in an appropriate volume of TE10/0.1.
(5) 1/5 volume of phenol chloroform, and isoamyl alcohol at 25:24:1 were added, mixed well and a resulting mixture was centrifuged at 13000 rpm for 30 min to remove the organic phase.
(6) Step (4) was repeated (usually 2-3 times) until no more white precipitate appears at the interface of the two phases.
(7) 1/5 volume of chloroform isoamyl alcohol at 24:1 was added, and mixed well, and a resulting mixture was centrifuged to remove the organic phase.
(8) 1/10 volume of 3 M NaAc pH=5.2 was added; 2.5 volumes of pre-cooled anhydrous ethanol was added.
(9) A resulting mixture was mixed well and left to stand at −20° C. overnight.
(10) Centrifugation was performed to collect the precipitate, the precipitate was left to stand until no ethanol odor existed, and dissolved in an appropriate volume of sterilized water, the UV absorbance was detected and the A260/A280 ratio was recorded.
   3. Plasmid Double Digestion Using Two Restriction Endonucleases to Obtain the Target dsDNA

23 μL of deionized water, 5 μL of 10×NEB cutsmart buffer (1×NEB cutsmart buffer, 50 mM KAC, 20 mM Tris-HAC, 10 mM Mg (AC)₂, 100 g/ml BSA, pH=7.9 at 25° C.), 20 μL of plasmid solution (concentration of 200 ng/L), 2 μL Kpn I (20 U), and 2 μL BamH I (20 U) were added to a 200 μL PCR tube and mixed well. The reaction was placed in a water bath at 37° C. for 4 h. The enzymatic efficiency was detected by 8% native-PAGE. The optimal plasmid concentration for the plasmid double digestion was 1000 ng/L.

4. Isolation and Purification of Target ssDNA
(1) The two single strands (wtTel23c and wtTel34) of the target ds DNA were separated using denaturing urea PAGE with a volume concentration of 12%. The electrophoresis was carried out in a JY-CX2B large vertical electrophoresis tank (35 cm×45 cm) with a constant power of 75 W for 8 h. The electrophoresis experiments were carried out in a biochemical incubator at a constant temperature of 30° C.
(2) Bands of target ssDNA were cut off and the gel block was threaded through a syringe and crushed into fine particles. An appropriate volume of TE10/0.1 was added and the DNA was immersed overnight in a 50° C. water bath (or eluted using an electroelution device).
(3) The soaked liquid was filtered through a 0.2 m filter membrane (or the liquid in the tank was aspirated directly if electroelution was used), then concentrated through a 3 K ultrafiltration tube, and ultrafiltered with water for several times to ultimately obtain the ssDNA. The ssDNA with natural abundance prepared in this experiment was named as NA-wtTel23c, and ¹⁵N-labeled ssDNA was named as ¹⁵N-wtTel23c.
(4) Denaturing urea PAGE was conducted.

### Example 2

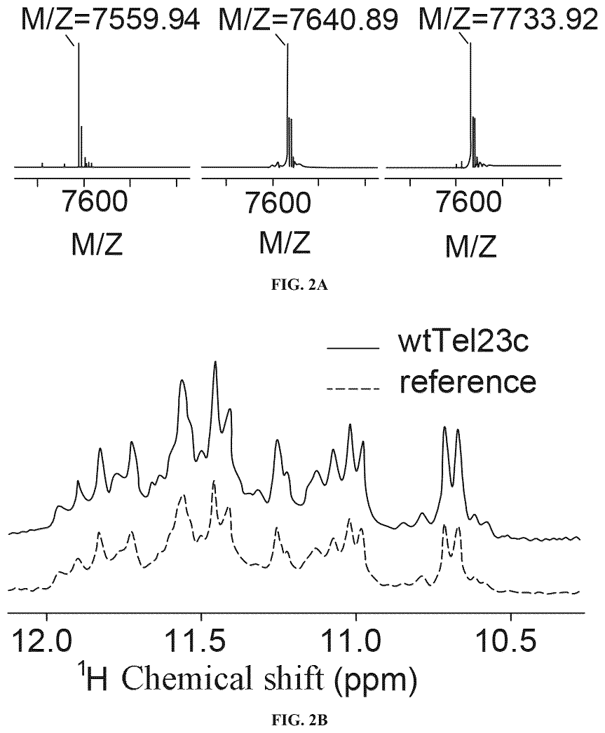

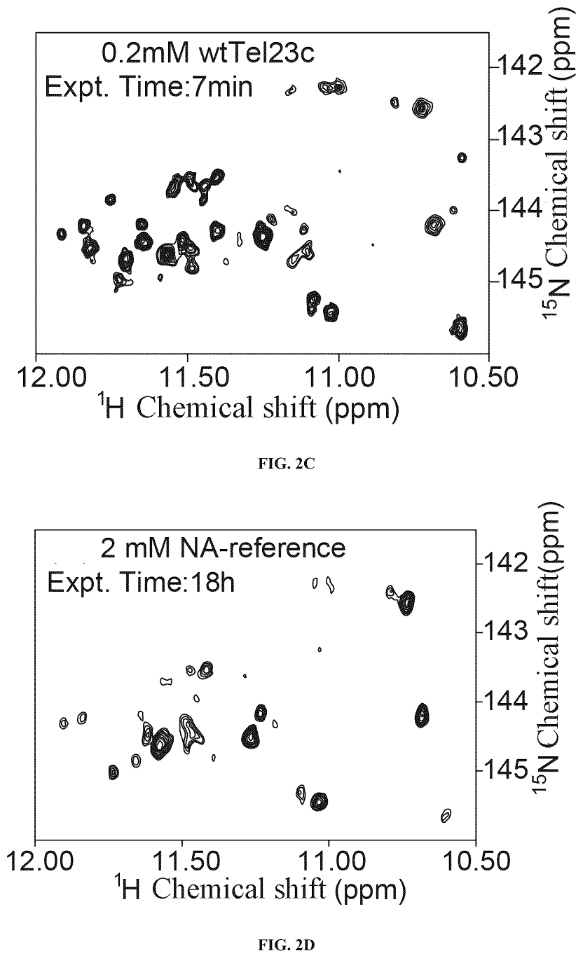

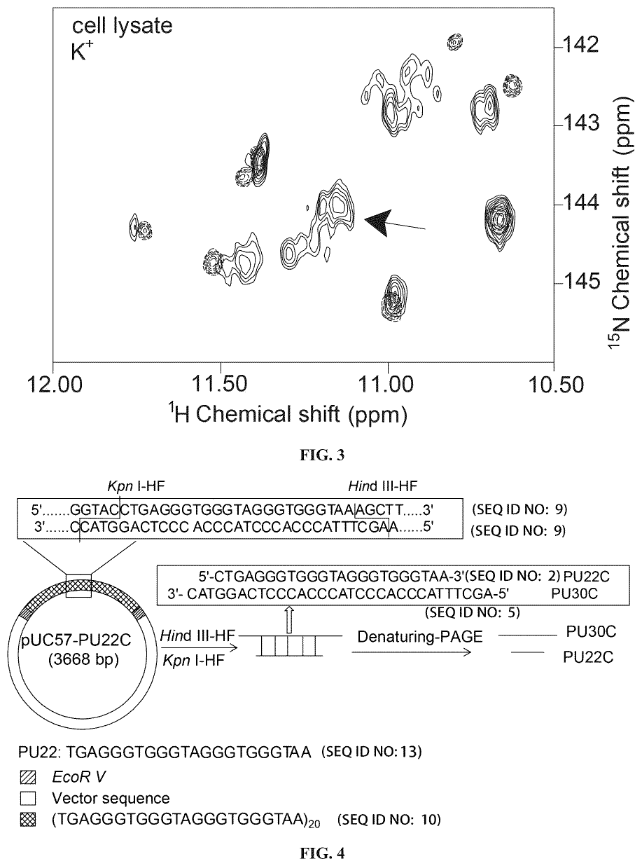

To verify that the target ssDNA (wtTel23c) obtained by E. coli amplification was consistent with chemically synthesized ssDNA samples of the same sequence, the target ssDNA was characterized using mass spectrometry, 2D ¹H-NMR, and 2D ¹H-¹⁵N NMR methods. High-performance liquid chromatography-mass spectrometry (LC-MS) coupling technique was used to determine the molecular weight of wtTel23c with natural abundance (NA-wtTel23c) synthesized in this experiment as well as the ¹⁵N labeling rate of ¹⁵N-wtTel23c. The experimental results are shown in FIGS. 2A to 2C. FIGS. 2A-2C are the mass spectra of the solid-phase synthesized reference sample (REFERENCE), the non-labeled wtTel23c (NA-wtTel23c) prepared in this experiment, and the ¹⁵N-labeled wtTel23c (¹⁵N-wtTel23c) prepared in this experiment, respectively, which showed that the relative molecular weight of solid-phase synthesized REFERENCE sample was 7559.94, while that of NA-wtTel23c with the same sequence was 7640.89. The difference in relative molecular weight between the two was 80.95. This was due to the fact that the 5′ and 3′ termini of the solid-phase synthesized wtTel23c ended with a hydroxyl group, while NA-wtTel23c was obtained by means of restriction endonuclease, in which the 5′ terminus ended with a phosphate group and the 3′ terminus ended with a hydroxyl group. Compared with the reference sample synthesized in solid phase, the molecular composition of NA-wtTel23c had one more P atom, three more O atoms and one more H atom. The sum of the relative atomic masses of these atoms was 79.97, so the molecular weight of NA-wtTel23c synthesized by biosynthesis method was in accordance with the expectation.

The relative molecular weight of ¹⁵N-wtTel23c was 7733.92, which was 93.02 higher than that of NA-wtTel23c. This is due to the fact that most of the nitrogen atoms (99.6%) in the sequence of NA-wtTel23c are ¹⁴N, whereas most of the nitrogen atoms in the sequence of ¹⁵N-wtTel23c are ¹⁵N atoms, and the number of nitrogen atoms contained in wtTel23c sequence is 97. The difference between the relative molecular weights of NA-wtTel23c and ¹⁵N-wtTel23c are theoretically 96.61, and the measured difference between the two is 93.02, which indicates that the labeling rate of the ¹⁵N-wtTel23c sequence is 96.28%. The formula for the calculation of the labeling rate of ¹⁵N refers to Equation 1.

$${}^{15} {N}_{labelling rate} = \frac{Mr ( {}^{15} N - wfTel 23 c ) - Mr ( NA - wfTel 23 c )}{N \times 996 \%} \qquad Equation 1$$

Note: N in Equation 1 indicates the number of nitrogen atoms in the wtTel23c sequence, i.e., 97.

NMR technique with atomic resolution was used to characterize the stacking of G bases in G4 formed by ssDNA folding obtained in this experiment. The results are shown in FIG. 2B. The samples (wtTel23c) obtained in this experiment as well as the reference sample (reference) showed 12 high-resolution signal peaks and some small peaks in the iminoproton region at 10-12 ppm, which indicates that the fine structure of the ssDNA folded to form G4 obtained in this experiment is consistent with that of the reference sample. ¹H-¹⁵N 2D sfHMQC is a fast 2D spectral acquisition technique that is widely used in the study of biological macromolecules. The technique can significantly improve the signal-to-noise ratio per unit time for heteronuclear-related experiments. The signals of NA-wtTel23c and ¹⁵N-wtTel23c samples were acquired using the sfHMQC technique. For ¹⁵N-labeled ssDNA samples with concentrations as low as 0.2 mM, high-resolution ¹H-¹⁵N sfHMQC 2D spectra were obtained in only 7 min (FIG. 2C), whereas for NA-wtTel23c samples with natural abundance and concentrations as high as 2.0 mM, the acquisition of ¹H-¹⁵N 2D sfHMQC spectra still required 18 h under the same spectrum acquisition conditions (FIG. 2D). This indicates that ¹⁵N-labeled DNA samples may improve the spectral sensitivity of ¹H-¹⁵N 2D spectra. Improving the signal-to-noise ratio per unit time lays the foundation for the study of complex biological systems.

There were significant differences in the ¹H-¹⁵N 2D sfHMQC spectra of the wtTel23c-TMPyP4 complexes obtained under dilute solution conditions versus a cell-like environment (as shown in FIGS. 2A-2D). As in the spectra obtained in a cell-like environment, the G21 signal indicated by the arrow was clearly retained while it did not appear in dilute solution conditions. This indicates that TMPyP4 binds to telomere G4 in a significantly different manner in the cell-like environment than in the dilute solution condition.

### Example 3 Preparation of ¹⁵N-Labeled Human c-Myc Promoter c-Myc ssDNA Sequence

The experimental principle of plasmid design is shown in FIG. 4, with c-Myc promoter DNA as the target sequence (5′-ctgagggtgggtagggtgggtaa-3′, SEQ ID NO:3), which is named as PU22C.

In order to improve the yield of target DNA, two different restriction sites, Kpn I-HF and Hind III-HF, were added to the 5′ and 3′ ends of the target sequence respectively to obtain the repeating unit, and the nucleotide sequence of the repeating unit was set forth in SEQ ID NO: 9, specifically: 5′-ggtacctgagggtgggtagggtgggtaaagctt-3′. The repeating unit was repeated 20 times in tandem to obtain the fusion sequence (nucleotide sequence is set forth in SEQ ID NO: 10, specifically:

GGTACCTGAGGGTGGGTAGGGTGGGTAAAGCTTGGTACCTGAGGGTGGGTAGGGTG
GGTAAAGCTTGGTACCTGAGGGTGGGTAGGGTGGGTAAAGCTTGGTACCTGAGGGTG
GGTAGGGTGGGTAAAGCTTatgttcagaatgaaactcatggaaacacttaaccagtgcataaacgctggtcatgaaatgac
gaaggctatcgccattgcacagtttaatgatgacagcccggaagcgaggaaaataacccggcgctggagaataggtgaagcagcggattt
agttggggtttcttctcaggctatcagagatgccgagaaagcagggcg(linker)GGTACCTGAGGGTGGGTAGGG
TGGGTAAAGCTTGGTACCTGAGGGTGGGTAGGGTGGGTAAAGCTTGGTACCTGAGGG
TGGGTAGGGTGGGTAAAGCTTGGTACCTGAGGGTGGGTAGGGTGGGTAAAGCTTtcacg
gatgggaaccagatcttcatattcatgcagaagacactctcctgcctttctatcttggggaaaaggacgatgtcacttatgcaataaagccca
cttgctggccggggcttgacattattccttcctgtctggctctgcaccgtattgaaactgagttaatgggcaaatttgatgaaggtaaactgc
ccaccgatc(linker)GGTACCTGAGGGTGGGTAGGGTGGGTAAAGCTTGGTACCTGAGGGTGG
GTAGGGTGGGTAAAGCTTGGTACCTGAGGGTGGGTAGGGTGGGTAAAGCTTGGTACC
TGAGGGTGGGTAGGGTGGGTAAAGCTTgttccacttgtatcgtcggtctgataatcagactgggaccacggtccca
ctcgtatcgtcggtctgattattagtctgggaccacggtcccactcgtatcgtcggtctgattattagtctgggaccacggtcccactcgtat
cgtcggtctgataatcagactgggaccacggtcccactcgtatcgtcggtctgattat(linker)GGTACCTGAGGGTGGGTA
GGGTGGGTAAAGCTTGGTACCTGAGGGTGGGTAGGGTGGGTAAAGCTTGGTACCTGA
GGGTGGGTAGGGTGGGTAAAGCTTGGTACCTGAGGGTGGGTAGGGTGGGTAAAGCT
Tttaatacgatacctgcgtcataattgattatttgacgtggtttgatggcctccacgcacgttgtgatatgtagatgataatcattatcactt
tacgggtcctttccggtgatccgacaggttacggggcggcgacctcgcgggttttcgctatttatgaaaattttccggtttaaggcgtttccg
ttcttcttcgtcata(linker)GGTACCTGAGGGTGGGTAGGGTGGGTAAAGCTTGGTACCTGAGGGTG
GGTAGGGTGGGTAAAGCTTGGTACCTGAGGGTGGGTAGGGTGGGTAAAGCTTGGTAC
CTGAGGGTGGGTAGGGTGGGTAAAGCTT.

Irrelevant sequences were added for ligations between the target sequences in every four repeats in the fusion sequence, and the fusion sequence was constructed into the high-copy vector pUC57. Asymmetric double-stranded DNA structures were generated by Kpn I-HF and Hind III-HF double digestion, and two ssDNAs of unequal lengths, PU22C: 5′-ctgagggtgggtagggtgggtaa-3′ (SEQ ID NO: 2); PU30C 5′-agctttacccaccctacccaccctcaggtac-3′ (SEQ ID NO: 5), were obtained by separation using denaturing urea PAGE, respectively. The tandemly duplicated target DNA sequences as well as linker sequences (1460 bp) were ligated to the vector pUC57 through sticky ends generated by EcoRV single enzyme digestion to construct the plasmid pUC57-PU22C.

### Methods

(i)¹⁵N-Pu22C Ssdna Preparation:

### 1. Amplification of Stable Isotope-Labeled Plasmids Using Labeled M9 Medium

The monoclonal colonies were selected and cultured in 5 mL of TB medium (37° C.). After 5 h, 5 mL of the E. coli solution was transferred to 50 mL of TB medium. After 3 h, the E. coli cells were collected by centrifugation, and then the E. coli cells were transferred to 150 mL of M9 medium containing ¹⁵NH₄Cl and cultured overnight. 50 mL of the culture solution from the overnight culture was added to 400 mL of fresh M9 medium containing ¹⁵NH₄Cl. When the OD600=1.0, the E. coli cells were collected by centrifugation, and washed by STE buffer at 37° C. The chloramphenicol solution with a final concentration of 11.5 mg/mL was added for incubation at 37° C. for 9 h. The E. coli cells were collected by centrifugation and washed with STE buffer for purification.

### 2. Plasmid Mass Extraction and Purification

Since the mass extraction plasmid kits available on the market could not meet the experimental requirements, the buffer solution required for mass extraction of plasmids was prepared according to the recipe in the Molecular Cloning Book (3rd edition).

3. Plasmid Double Digestion Using Two Restriction Endonucleases to Obtain the Target dsDNA

23 μL of deionized water, 5 μL of 10×NEB cutsmart buffer (1×NEB cutsmart buffer, 50 mM KAC, 20 mM Tris-HAC, 10 mM Mg (AC)₂, 100 g/ml BSA, pH=7.9 at 25° C.), 20 μL of plasmid solution (concentration of 200 ng/L), 2 μL Kpn I (20 U), and 2 μL Hind III (20 U) were added to a 200 μL PCR tube and mixed well. The reaction was carried out in a water bath at 37° C. for 4 h. The digestion efficiency was detected by 8% native-PAGE. The optimal plasmid concentration for the plasmid double digestion reaction was 1000 ng/L.

4. Isolation and Purification of Target ssDNA
(1) The two single strands of target dsDNA (wtTel23c and wtTel34) were separated using 12% denaturing urea PAGE. The electrophoresis was carried out in a JY-CX2B large vertical electrophoresis tank (35 cm×45 cm) with a constant power of 75 W for 8 h. The electrophoresis experiments were carried out in a biochemical incubator at a constant temperature of 30° C.
(2) Bands of target ssDNA were cut off and the gel block was threaded through a syringe and crushed into fine particles. An appropriate volume of TE10/0.1 was added and the DNA was immersed overnight in a 50° C. water bath (or eluted using an electroelution device).
(3) The soaked liquid was filtered through a 0.2 m membrane (or directly aspirated from the tank if electroelution was used), then concentrated through an ultrafiltration tube with a cutoff of 3 KDa, and the ssDNA was finally obtained by exchanging the buffer with water ultrafiltration for several times. The ssDNA with natural abundance prepared in this example was named as NA-PU22C, and ¹⁵N-labeled ssDNA was named as ¹⁵N-PU22C.

### (ii) Preparation of G4s

(1) PU22C (SEQ ID NO: 2): 5′-ctgagggtgggtagggtgggtaa-3′
(2) K⁺ buffer solution: 2.5 mM K₃PO₄, 7.5 mM KCl, pH=6.0
(3) Preparation of ¹⁵N-labeled PU22C: ¹⁵N-labeled ssDNA was prepared by the above method, dissolved in K⁺ buffer solution, subjected to heating at 95° C. for 10 min, gradually cooled to 4° C., and equilibrated at 4° C. for 0.5 h to form a stable G4s structure.
(4) Preparation of unlabeled G4s: purchased DNA powder was dissolved in deionized water and ultrafiltered three times to remove inorganic salt ions as well as organic solvents, such as triethylamine, from the sample as much as possible. Then the DNA was subjected to elevated temperature annealing under the condition of K⁺ buffer solution, heated at 95° C. for 10 min, gradually cooled to 4° C., and equilibrated at 4° C. for 0.5 h to form stable G4s.

### Example 4 Nuclear Magnetic Resonance Experiment

1. ¹H-¹⁵N 2D sfHMQC Experiments

To 200 μL of ¹⁵N-labeled PU22C prepared in Example 3 prepared ahead of time, 10% v/v heavy water (D₂O) was added, with a total sample volume of 220 μL, resulting in a final concentration of the DNA sample of 0.2 mM. The sample was loaded into a shigemi NMR tube, and NMR experiments were performed on a 700 MHz Bruker spectrometer equipped with an ultra-low temperature probe. The sampling temperature was 298 K. The ¹H-¹⁵N 2D sfHMQC spectra were sampled at 400 points in the direct dimension and 40 points in the indirect dimension, with a spectral width of 12 ppm and a center position at 10.5 ppm in the direct dimension, and a spectral width of 6 ppm and a center position at 144 ppm in the indirect dimension. The cumulative number of scans was 16. The NMR data were acquired using Topspin software and processed using CARAR software.

2. ²JH_{N} ¹H-¹⁵N HSQC Selectivity Experiments

NMR experiments were performed on a 700 MHz Bruker spectrometer equipped with an ultra-low temperature probe. The sampling temperature of the experiment was 298 K. The two-dimensional ¹H-¹⁵N HSQC spectra were sampled with 1408 points in the direct dimension and 128 points in the indirect dimension, and the spectral width in the direct dimension was 20 ppm. The spectral centers were located depending on the region selected. The cumulative number of scans was 80. ¹⁵N-band-selective heteronuclear single quantum coherence (HSQC) pulse sequence (¹⁵N-band-selective ¹H-¹⁵N HSQC) was conducted. ¹⁵N-selective pulse excitation was realized in a certain frequency range by means of soft pulse or shape pulse (Q3 shape pulse).

### Example 5

(i) Characterization of the Preparation Process of ¹⁵N-PU22C ssDNA Prepared in Example 3

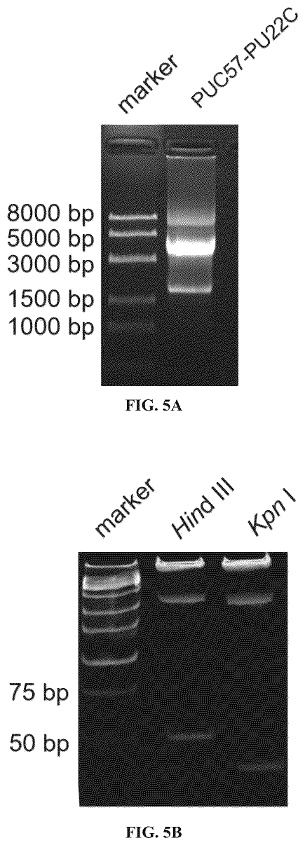

The crude extract of plasmid was obtained by alkaline lysis, and the crude extracted plasmid was purified by phenol-chloroform and other steps. The purified plasmid was detected by agarose gel electrophoresis, and the results are shown in FIG. 5A. The purified plasmid showed three bands, and the apparent molecular weights from smallest to largest indicated the super-helical plasmid, the cyclic plasmid, and the linear plasmid, respectively. The middle band is the highest in content, which indicates that the purified plasmid (pUC57-PU22C) exists mainly in the form of a cyclic form. The pUC57-PU22C showed an apparent molecular weight on the gel that was in accordance with expectations. Optimization of the double digestion conditions was performed, and the experimental results showed that the restriction endonucleases Kpn I-HF and Hind III-HF could simultaneously digest pUC57-PU22C. In addition, during the optimization of the experiment, it was found that after the plasmid was digested by Kpn I-HF, the next step of digestion with Hind III-HF failed; however, the target dsDNA was efficiently obtained when the plasmid was first digested by Hind III-HF, and then by Kpn I-HF (e.g., FIG. 5B). This may be attributed to the different binding ability of the two enzymes to dsDNA. The target dsDNA produced after double digestion was purified by denaturing urea PAGE (35 cm×45 cm), and the gel was cut to recover the target ssDNA bands, and the purified ssDNA samples were obtained by an electroelution method. The ssDNA sample with the same sequence prepared by solid phase synthesis method was used as a reference and detected by 12% acrylamide gel electrophoresis, and the results showed that the molecular weight of the PU22C prepared in this example was the same as that of the PU22C synthesized in solid phase.

(ii) PU22C Interacts with CX5461

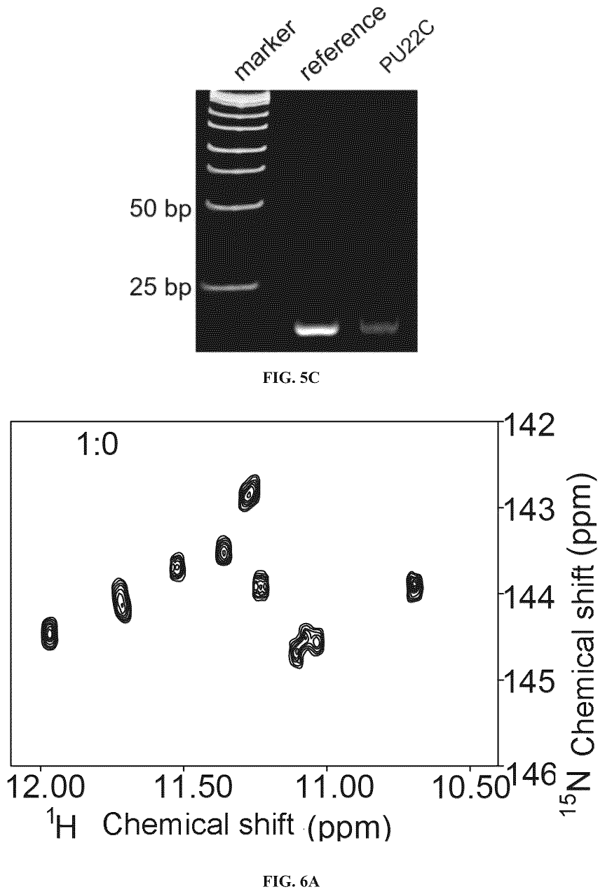

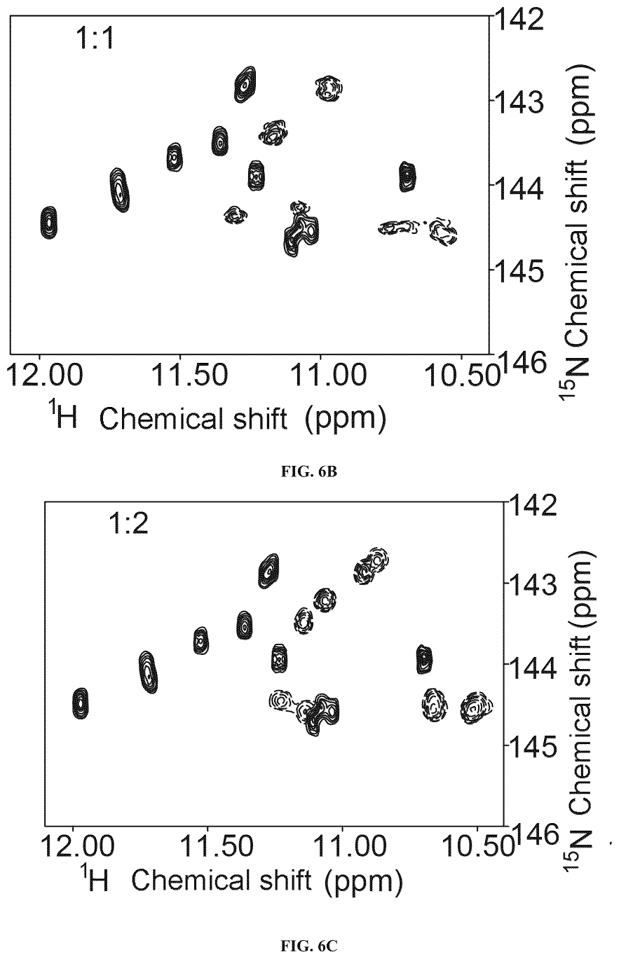

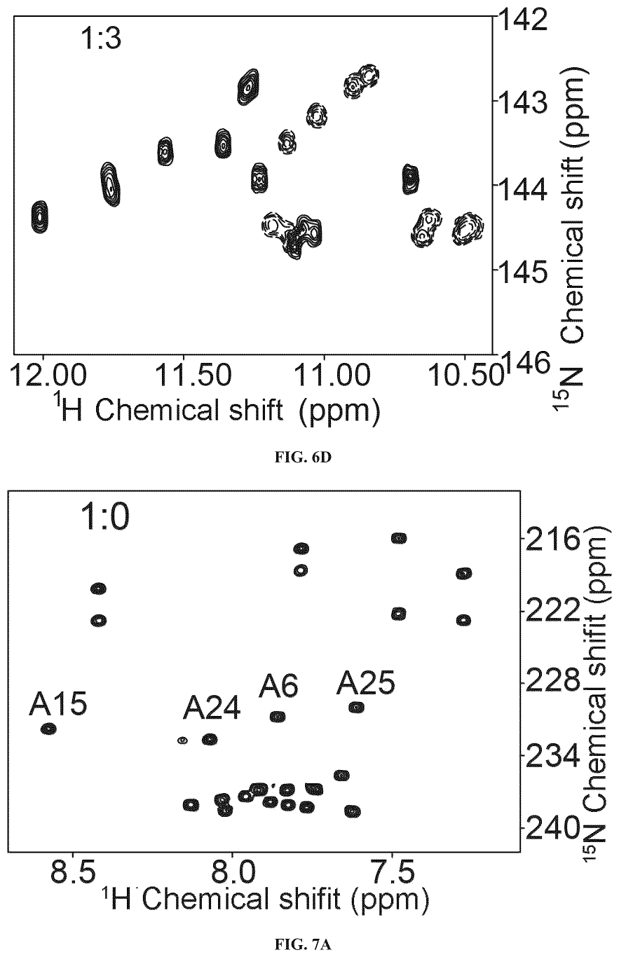

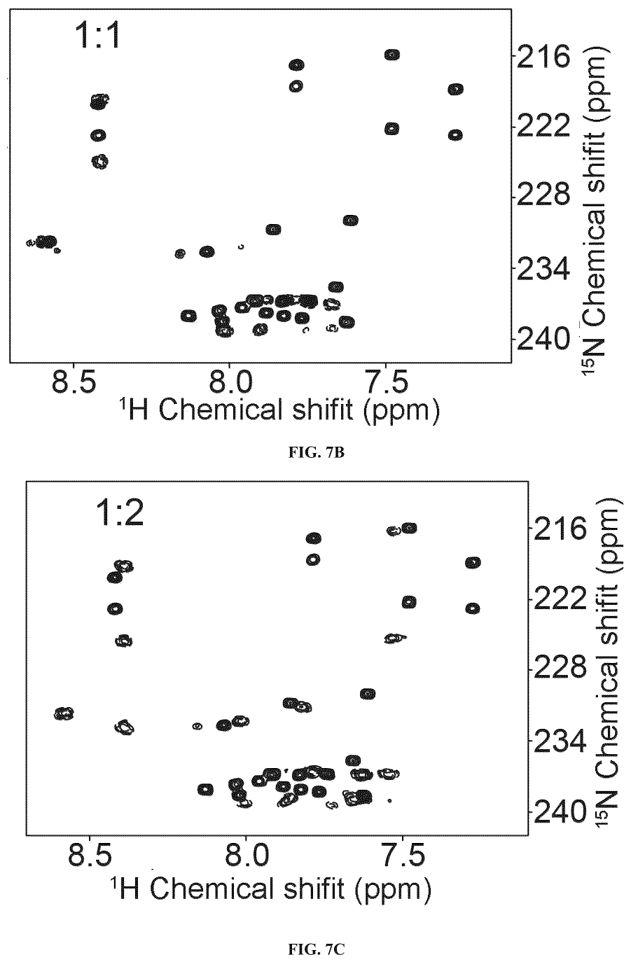

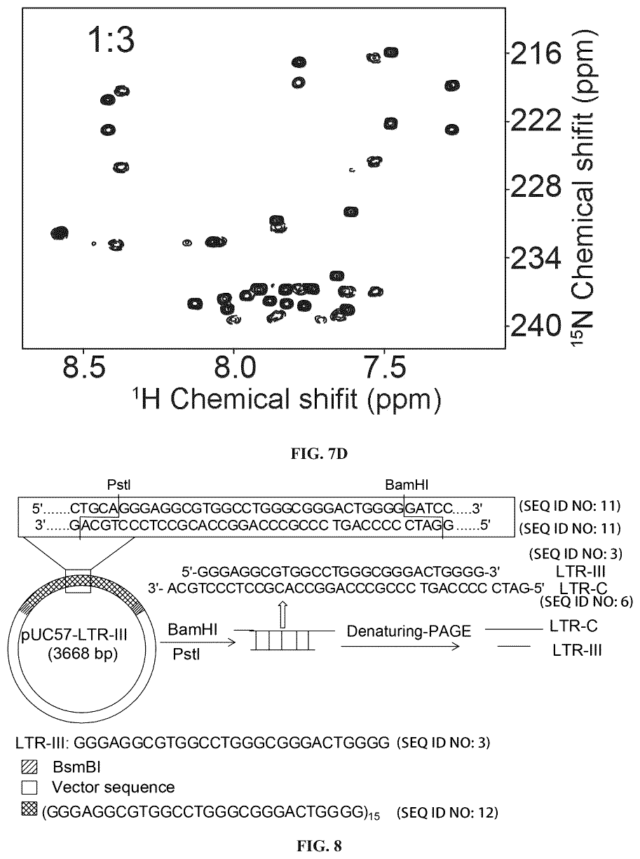

PU22C is a cancer-associated c-Myc promoter G4s. The clinical drug CX5461 can specifically bind to G4s and is currently in phase I clinical trial against BRCA1/2-deficient tumors, but the structure of the CX5461-G4s complex is unknown. Therefore, it is scientifically important to explore the interaction between CX5461 with G4s. PU22C was titrated with CX5461 at different molar ratios. The 1D ¹H-NMR spectra of PU22C in the bound state had poor resolution and limited information about the complex obtained. The ¹H-¹⁵N 2D sfHMQC were utilized to characterize the interaction between CX5461 and PU22C. As shown in FIG. 6A-2D, the 2D spectra could capture the course of the spectral signal. At a low molar concentration (PU22C:CX5461=1:1), the spectral signal broadened, and with the increase of CX5461 concentration (PU22C:CX5461=1:2), the spectral signal enhanced. This indicates that the binding of PU22C to CX5461 on a intermediate time scale of NMR. When the ratio of PU22C:CX5461 is increased to 1:3, the spectra no longer changed significantly, indicating that the interaction of PU22C with CX5461 reached the titration endpoint at 1:2. At the end point of the titration, the spectra showed eight independent iminoproton signals, which was possibly due to the signal overlap. The same characterization was carried out using ²JH_{N} ¹H-¹⁵N sfHMQC NMR spectra and the results are shown in FIGS. 7A-7D. The signals of the G and A bases changed significantly after they bound to the drug, where the signal of A15 did not change significantly before and after the titration, and the signals of the other A bases (A6, A24, and A25) were significantly shift. There was significant shifts on signals of other A bases (A6, A24 and A25), indicating that A15 was not involved in the binding site of CX5461, which was located in the loop region of the G4s structure. However, the other A bases are located in the 5′ and 3′ ends of the G4s, respectively. This indicates that CX5461 bound to the 5′ and 3′ ends of PU22C in a 1:2 binding ratio. This is similar to the binding pattern of quindoline-i to c-Myc G4s (see [Liu, W., Lin, C., Wu, G., Dai, J., Chang, T. C. and Yang, D. (2019) Structures of 1:1 and 2:1 complexes of BMVC and MYC promoter G-quadruplex reveal a mechanism of ligand conformation adjustment for G4-recognition. (Nucleic Acids Res, 47, 11931-11942.]).

In the traditional structure determination method of G4s, the protons inside the bases as well as the proton signals of adjacent bases are analyzed by correlating the H8 of the A and G bases with the protons on the sugar ring through Nuclear Overhauser Effect (NOE) signals. However, large number of signals, difficulties in signal attribution, or partial signal losses of NOE due to spatial distance or angle problems, prevents the next step of correlation. This is the bottleneck problem encountered in the current NMR technology for analyzing G4 structure. The ¹³N-labeled human c-Myc promoter c-Myc ssDNA sequence of the present disclosure realizes selective excitation of signals in specific proton regions by the method of ¹⁵N spectra editing, which allows obtaining proton signals (such as proton signals of iminos) in specific regions, drastically simplifying the spectral signals, and improving the efficiency of spectral signal resolution.

### Example 6 Preparation of ¹⁵N-Labeled HIV ssDNA Sequence LTR-III

The experimental principle of plasmid design is shown in FIG. 8, with HIV LTR-III DNA (5′-gggaggcgtggcctgggcgggactgggg-3′, SEQ ID NO: 3) as the target sequence, which is named as LTR-III.

In order to improve the yield of target DNA, two different restriction sites, Pst I-HF and Hind III-HF, were added to the 5′ and 3′ ends of the target sequence respectively to obtain the repeating unit, and the nucleotide sequence of the repeating unit is set forth in SEQ ID NO: 11 (5′-ctgcagggaggcgtggcctgggcgggactgggggatcc-3′). 15 repeating units were repeated in tandem to obtain the fusion sequence (the nucleotide sequence is set forth in SEQ ID NO: 12, specifically:

CTGCAGGGAGGCGTGGCCTGGGCGGGACTGGGGGATCCCTGCAGGGAGGCGTGG
CCTGGGCGGGACTGGGGGATCCCTGCAGGGAGGCGTGGCCTGGGCGGGACTGGGGG
ATCCatgttcagaatgaaactcatggaaacacttaaccagtgcataaacgctggtcatgaaatgacgaaggctatcgccattgcacagttt
aatgatgacagcccggaagcgaggaaaataacccggcgctggagaataggtgaagcagcggatttagttggggtttcttctcaggctatca
gagatgccgagaaagcagggcg(linker)
CTGCAGGGAGGCGTGGCCTGGGCGGGACTGGGGGATCCCTGCAGGGAGGCGTGG
CCTGGGCGGGACTGGGGGATCCCTGCAGGGAGGCGTGGCCTGGGCGGGACTGGGGG
ATCCtcacggatgggaaccagatcttcatattcatgcagaagacactctcctgcctttctatcttggggaaaaggacgatgtcacttatgca
ataaagcccacttgctggccggggcttgacattattccttcctgtctggctctgcaccgtattgaaactgagttaatgggcaaatttgatgaa
ggtaaactgcccaccgatc (linker)
CTGCAGGGAGGCGTGGCCTGGGCGGGACTGGGGGATCCCTGCAGGGAGGCGTGG
CCTGGGCGGGACTGGGGGATCCCTGCAGGGAGGCGTGGCCTGGGCGGGACTGGGGG
ATCCgttccacttgtatcgtcggtctgataatcagactgggaccacggtcccactcgtatcgtcggtctgattattagtctgggaccacggt
cccactcgtatcgtcggtctgattattagtctgggaccacggtcccactcgtatcgtcggtctgataatcagactgggaccacggtcccactc
gtatcgtcggtctgattat (linker)
CTGCAGGGAGGCGTGGCCTGGGCGGGACTGGGGGATCCCTGCAGGGAGGCGTGG
CCTGGGCGGGACTGGGGGATCCCTGCAGGGAGGCGTGGCCTGGGCGGGACTGGGGG
ATCCttaatacgatacctgcgtcataattgattatttgacgtggtttgatggcctccacgcacgttgtgatatgtagatgataatcattatca
ctttacgggtcctttccggtgatccgacaggttacggggggcgacctcgcgggttttcgctatttatgaaaattttccggtttaaggcgtttc
cgttcttcttcgtcata (linker)
CTGCAGGGAGGCGTGGCCTGGGCGGGACTGGGGGATCCCTGCAGGGAGGCGTGG
CCTGGGCGGGACTGGGGGATCCCTGCAGGGAGGCGTGGCCTGGGCGGGACTGGGGG
ATCC),

and every four replicates of the target sequence in the fusion sequence were added with irrelevant sequence as linkers, and the fusion sequence was constructed into the high-copy vector pUC57. Asymmetric double-stranded DNA structures were generated by double digestion with Pst I-HF and Hind III-HF, and two ssDNAs of unequal lengths, 5′-gggaggcgtggcctgggcgggactgggg-3′ (LTR-III, SEQ ID NO: 3) and 3′-acgtccctccgcaccggacccgccctgaccccctag-5′ (LTR-C, SEQ ID NO: 6), were obtained by denaturing urea PAGE. The tandemly duplicated target DNA sequences as well as linker sequences (1370 bp) were ligated to the vector pUC57 through sticky ends generated by BsmBI single enzyme digestion to construct a plasmid, which was named as pUC57-LTR-III.

### Methods

(i)¹⁵N-Ltr-III ssDNA Preparation:

### 1. Amplification of Stable Isotope-Labeled Plasmids Using Labeled M9 Medium

The monoclonal colonies were selected and cultured in 5 mL of TB medium (37° C.). After 5 h, 5 mL of the E. coli solution was transferred to 50 mL of TB medium. After 3 h, the E. coli cells were collected by centrifugation, and then the bacteria were transferred to 150 mL of M9 medium containing ¹⁵NH₄Cl and cultured overnight. 50 mL of bacteria solution cultured overnight was added to 400 mL of fresh M9 medium containing ¹⁵NH₄ Cl. When the OD=1.0, the bacteria were collected by centrifugation, and washed with STE buffer. The chloramphenicol was added at the final concentration of 11.5 mg/mL for incubation at 37° C. for 9 h. The E. coli cells were collected by centrifugation and washed with STE buffer for purification.

### 2. Plasmid Mass Extraction and Purification

Since the mass extraction plasmid kits available on the market could not meet the experimental requirements, the buffer solution required for mass extraction of plasmids was prepared according to the recipe in the Molecular Cloning Book (3rd edition).

3. Plasmid Double Digestion Using Two Restriction Endonucleases to Obtain the Target dsDNA

23 μL of deionized water, 5 μL of 10×NEB cutsmart buffer (1×NEB cutsmart buffer, 50 mM KAc, 20 mM Tris-HAc, 10 mM Mg (Ac)₂, 100 g/ml BSA, pH=7.9 at 25° C.), 20 μL of plasmid solution (concentration of 200 ng/L), and 2 μL Kpn I (20 U), 2 μL Hind III (20 U) were added to a 200 μL PCR tube and mixed well. The reaction was carried out in a water bath at 37° C. for 4 h. The digestion efficiency was detected by 8% native-PAGE. The optimal plasmid concentration for the plasmid double digestion reaction was 1000 ng/L.

4. Isolation and Purification of Target ssDNA
(1) The two single strands (wtTel23c and wtTel34) of the target dsDNA were separated using 12% denaturing urea PAGE. The electrophoresis was carried out in a JY-CX2B large vertical electrophoresis tank (35 cm×45 cm) with a constant power of 75 W for 8 h. The electrophoresis experiments were carried out in a biochemical incubator at a constant temperature of 30° C.
(2) Bands of target ssDNA were cut off and the gel block was threaded through a syringe and crushed into fine particles. An appropriate volume of TE10/0.1 was added and the DNA was immersed overnight in a 50° C. water bath (or eluted using an electroelution device).
(3) The soaked liquid was filtered through a 0.2 m membrane (or the liquid in the tank was aspirated directly if electroelution was used), and then concentrated in an ultrafiltration tube with a molecular weight cut-off of 3 K. The ssDNA was finally obtained by changing the buffer with water ultrafiltration for several times. The ssDNA with natural abundance prepared in the present experiments was named as NA-LTR-III, and ssDNA labeled with ¹⁵N was named as 15N-LTR-III.

### (ii) Preparation of G4 Samples

(1) LTR-III (SEQ ID NO: 3): 5′-gggaggcgtggcctgggcgggactgggg-3′
(2) K⁺ buffer solution: 20 mM K₃PO₄, 70 mM KCl, pH=7.0
(3) Preparation of ¹⁵N-labeled G4 (LTR-III): ¹⁵N-labeled ssDNA was prepared by the method described above, dissolved in K⁺ buffer solution, heated at 95° C. for 10 min, gradually cooled to 4° C., and equilibrated at 4° C. for 0.5 h to form a stable G4 structure.
(4) Preparation of unlabeled G4: purchased DNA powder was dissolved in deionized water and ultrafiltered three times to remove as much as possible the inorganic salt ions as well as organic solvents, such as triethylamine, from the sample. Then a resulting product was subjected to annealing under the condition of K⁺ buffer solution with elevated temperature, heating at 95° C. for 10 min, gradual cooling to 4° C., and equilibrating at 4° C. for 0.5 h to form stable G4.

### Example 7 Nuclear Magnetic Resonance Experiments for ¹H-¹⁵N 2D sfHMQC

10% v/v heavy water (D₂O) was added to 200 μL of ¹⁵N-labeled LTR-III sample prepared ahead of time in Example 6, with a total sample volume of 220 μL and the final concentration of the DNA sample being 0.2 mM. The samples were loaded into a shigemi NMR tube, and NMR experiments were carried out on a 700 MHz Bruker spectrometer equipped with a cryo-probe. The sampling temperature of the experiment was 298 K. The ¹H-¹⁵N 2D sfHMQC spectra under dilute solution conditions had a direct dimensional sampling number of 400 and an indirect dimensional sampling number of 40, with a spectral width of 12 ppm and a spectral center position at 10.5 ppm in the direct dimension, and a spectral width of 6 ppm and a spectral center position at 144 ppm in the indirect dimension. The cumulative number of scans was 16. For the ¹H-¹⁵N 2D sfHMQC spectrum acquisition in a cellular environment, the number of scans increased to 2048. NMR data acquisition was performed using Topspin software and data processing was performed using CARA software.

### Example 8

1. ¹⁵N-Labeled LTR-III ssDNA Yields a Fingerprint of LTR-III G4 and its Interaction with Ligands

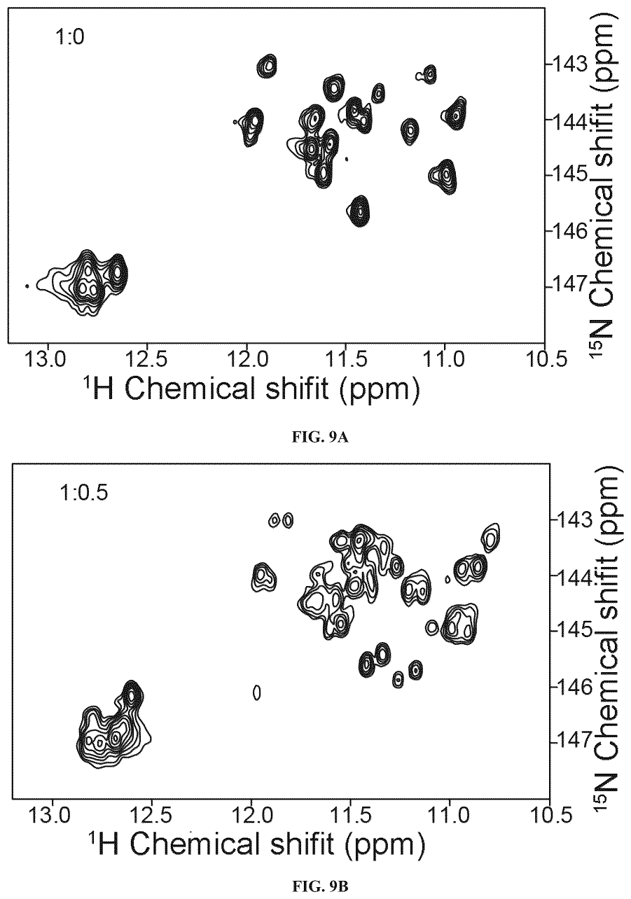

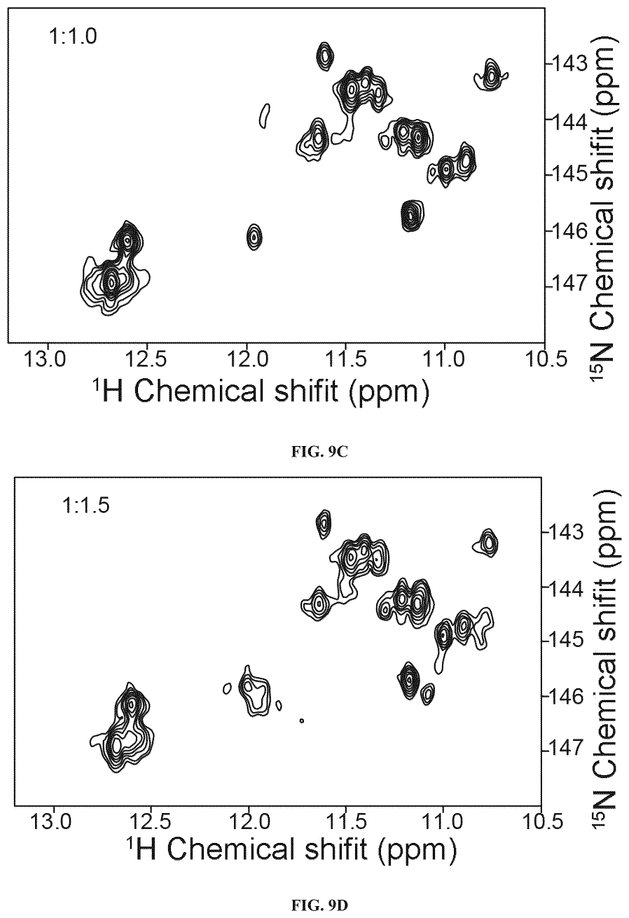

NMR characterization of LTR-III G4 structure is usually performed using bases at a single specific site for ¹⁵N-labeling, followed by a spectroscopic method of H1 proton signal-by-signal fingerprinting via ¹⁵N-filtering. For example, in the structure analysis of LTR-III G4, the 15 guanines of LTR-III G4 were each ¹⁵N-labeled at a single site, and then a spectroscopic method of obtaining a single H1 proton signal through ¹⁵N filtering was performed, and the 15 guanines of LTR-III G4 were thus individually designated. The advantage of this method is the single signal and clear designation. However, the obvious disadvantage is that samples multiple single site ¹⁵N-labeled are required for separate NMR spectra and the fingerprinting of LTR-III G4 cannot be obtained. In the ¹⁵N-labeled LTR-III G4 of the present disclosure, LTR-III G4 is uniformly labeled with the ¹⁵N, and a 2D NMR fingerprint of LTR-III G4 may be obtained in a single spectrum acquisition. The structure of LTR-III G4 is different from that of the conventional G4 in that there are three classical GC base pairs in addition to three layers of G-tetrad. In the present disclosure ¹⁵N-labeled LTR-III G4, high-resolution structural information may be obtained by the acquisition of 2D sfHMQC spectra, in which the process of the spectral signal changes may be observed. The results are shown in FIG. 9A. When the ligand Braco-19 (1:0) was not added to the system, the proton signals for Hoogeteen hydrogen bond N7-H1 . . . O6 formed by the GG non-canonical base pairing in the G4 (10-12 ppm) as well as proton signals for Watson-Crick hydrogen bond formed by canonical pairing of GC (12-13 ppm) could be clearly detected on the ¹H-¹⁵N sfHMQC spectra. Titration of the ligand Braco-19 was carried out under the same conditions, and the signals of the LTR-III G4 spectra changed significantly with the addition of Braco-19 at titration (molar) ratios ranging from 1:0.5 to 1:1.5 (FIGS. 9B-9D). When the titration ratio was 1:1.5, the spectral signal no longer changed significantly compared with the 1:1 spectra, indicating that LTR-III G4 and Braco-19 reached the endpoint of the titration at 1:1. The signals in the region of the three GC pairings (12-13 ppm) and the G4 region (10-12 ppm) in the spectra both changed significantly, suggesting that the ligand molecules were likely to bind to the interfacial junction between the G-tetrad and GC base pairing.

### 2. Effects of Complex Cellular Environment on LTR-III G4 Conformation Based on ¹⁵N-Labeled LTR-III G4 Study

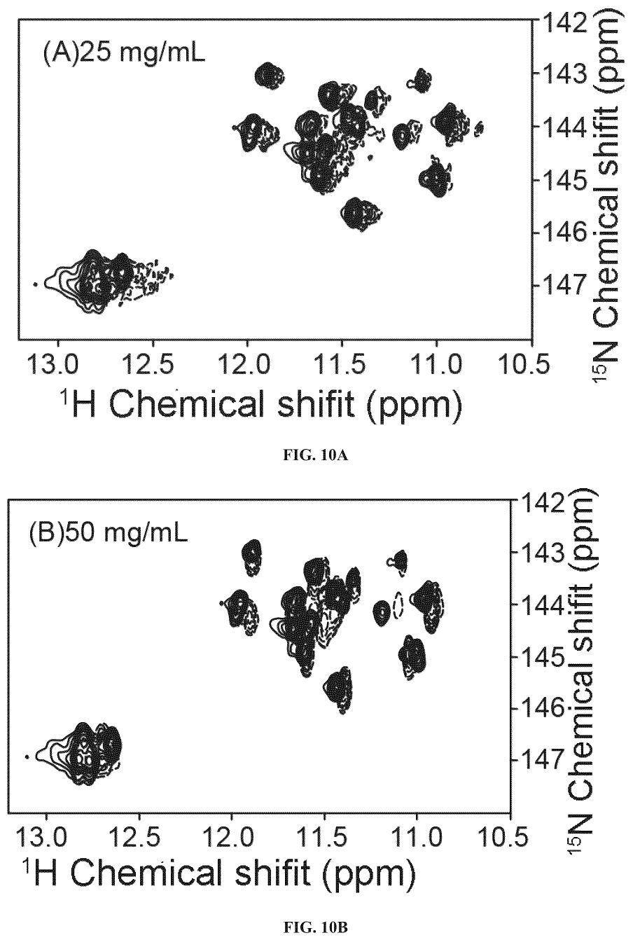

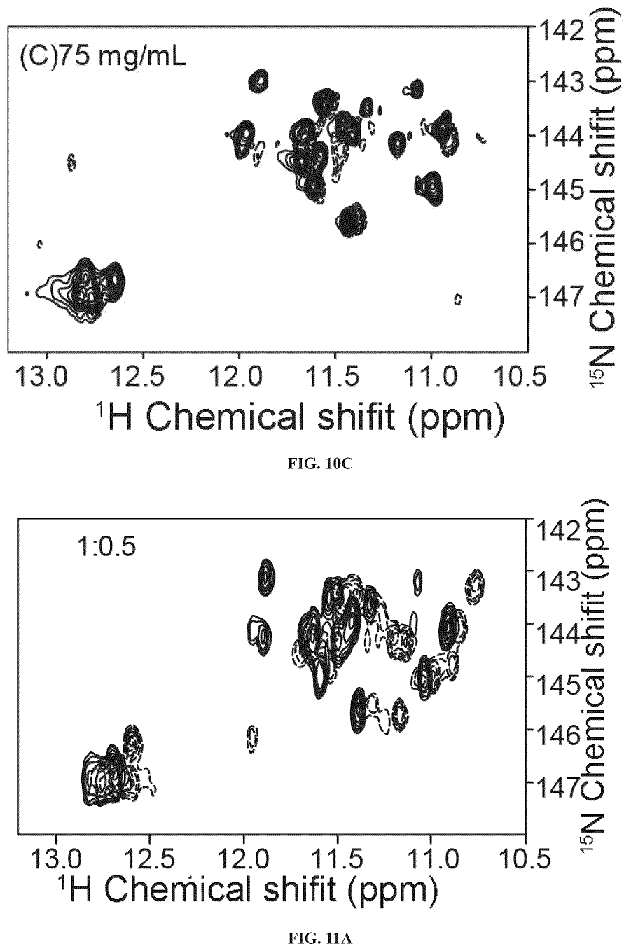

Under dilute solution conditions, it is possible for ligand molecules to bind to the interfacial junction of the G-tetrad and GC base pairing. However, the cellular environment of proteins, small molecules of metabolites, and crowded environments may have an effect on the conformation of LTR-III G4. Whether the unique conformation of LTR-III G4 exists in the cellular environment remains unknown. The ¹⁵N-labeled LTR-III G4 in the present disclosure has the advantage of uniform labeling with the ¹N, which allows direct access to structural information of LTR-III G4 in complex cellular environments. The fingerprints of LTR-III G4 in cell-like environments were collected at concentration gradients of cell extracts of 0 mg/mL (gray spectra in FIGS. 10A-10C), 25 mg/mL, 50 mg/mL, and 75 mg/mL (red spectra, FIGS. 10A-10C). As the concentration of the cell extract increased, the signal in the spectra did not change significantly. In the spectra at a suitable concentration of cell extract of 50 mg/mL (FIG. 10B), the signals of both the region of three GC pairings (12-13 ppm) and the G4 region (10-12 ppm) did not undergo significant changes, suggesting that the unique conformation of LTR-III G4 still existed in the cellular environment, which could be a potential special site of action for drug molecules targeting LTR-III G4. In addition, the spectral signals weakened or even disappeared at a cellular extract concentration of 75 mg/mL, which was attributed to the increase in nonspecific interactions with the increase in cellular extract concentration.

3. Direct Access to Obtain the Interaction of LTR-III G4 with Ligand Braco-19 in Complex Cellular Environments Based on ¹⁵N-Labeled LTR-III G4 Samples

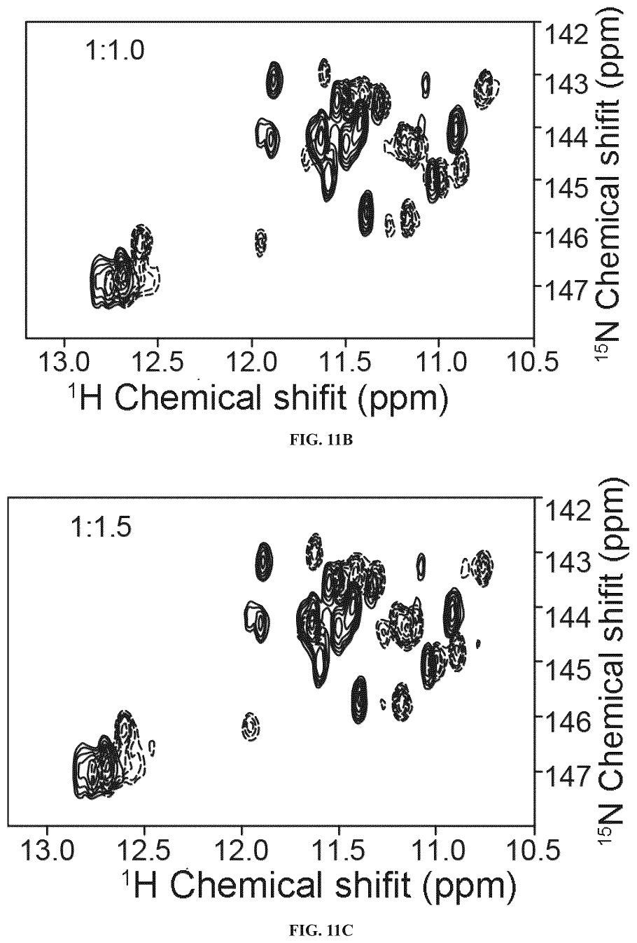

Studying the interaction between LTR-III G4 and the ligand Braco-19 in a cellular environment and obtaining information such as relevant binding sites are important guidelines for the rational design of G4 ligands. In-cell NMR technology is free from the interference of background signals, and may obtain the signals of the target molecules clearly under the complex cellular environment, which is suitable for the assessment of the interactions between G4 and ligands in the cellular environment. In order to obtain a better resolution of the spectra, the inventors chose to explore the interaction between LTR-III G4 and the ligand Braco-19 at a cell-like concentration of 50 mg/mL. The results are shown in FIGS. 11A-11C, where the gray background plot indicates the spectral signal of LTR-III G4 in the free state under the condition of cell concentration of 50 mg/mL; the red spectral plot indicates the spectral information of the complexes with different titration ratios under the condition of cell extract. With the increase of ligand concentration, the red spectral signal changed significantly, indicating that the ligand Braco-19 could selectively interact with LTR-III G4 in the environment of cell extract. When the titration ratio ranged from 1:0.5 to 1:1.5 (FIGS. 11A-11C), the red spectra no longer changed significantly when the titration ratio is 1:1.0, indicating that the end point of the titration was reached at a titration ratio 1:0. At the titration ratio of 1:0 (FIG. 11B), the information of the LTR-III G4 spectra in the bound state (red spectra) was merged with that of LTR-III G4 in the free state (black spectra), and it could be seen that the signals in both the region of three GC pairings (12-13 ppm) and the G4 region (10-12 ppm) were significantly changed, indicating that in the cellular environment, the ligand molecule was likely to bind to the interfacial junction between the G-tetrad and GC pairing. The study of ¹⁵N-labeled LTR-III G4 in the present disclosure may further demonstrate the value of targeting TR-III G4 at the interfacial junction of G-tetrad and GC pairing, and provide important guidance for the rational design of drugs targeting TR-III G4.

### Example 9 Preparation of Stable Isotope ¹³C-Labeled PU22C

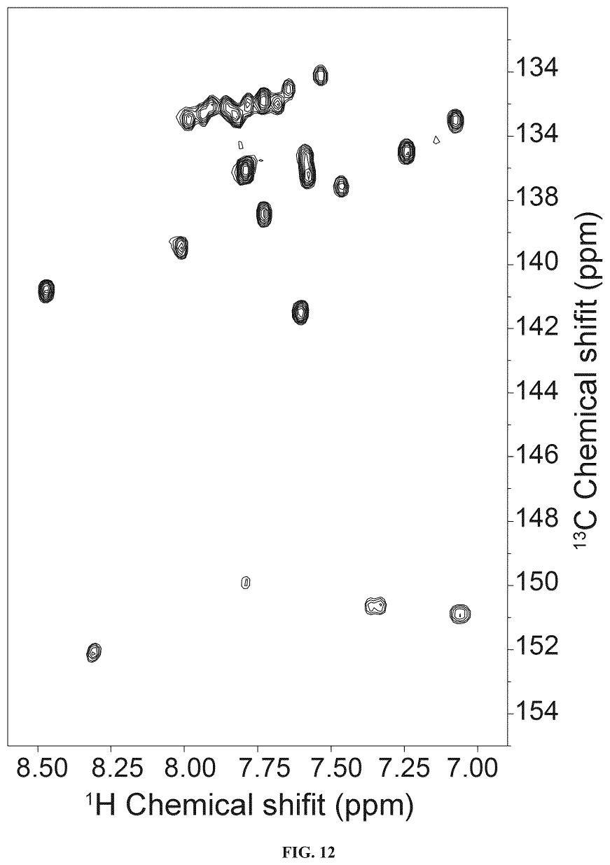

The preparation of stable isotope ¹³C-labeled PU22C was similar to the preparation of ¹⁵N-labeled PU22C in Example 3, except that the fresh M9 medium of ¹⁵NH₄Cl was replaced with fresh M9 medium with ¹⁵N—NH₄Cl and ¹³C-glucose as the sole nitrogen source and the sole carbon source, respectively. The ssDNA was purified in the same manner. The signals of the C—H bonds of various types of bases could be obtained by the acquisition of HSQC spectra signals, as shown in FIG. 12. Selective excitation of the ¹³C nucleus enabled the selective acquisition of CH signals, which may simplify the signals of the NMR spectra and facilitate the NMR studies of nucleic acids.

Although the present disclosure was described in detail by the above examples, it is only a part of the embodiments of the present disclosure, not all of the embodiments. One can obtain other embodiments according to the present embodiments without an inventive step, and these embodiments are all within the protection scope of the present disclosure.

What is claimed is:

1. A method for preparing stable isotope-labeled single-stranded DNA (ssDNA) by biosynthesis with Escherichia coli (E. coli), comprising the steps of:
1) adding a site of a first restriction endonuclease and a site of a second restriction endonuclease to 5′ and 3′ ends of a target sequence, respectively, to obtain a repeating unit; wherein the first restriction endonuclease and the second restriction endonuclease are different;
2) ligating the repeating unit obtained in step 1) in tandem to obtain a fusion sequence;
3) inserting the fusion sequence in step 2) into a high-copy vector to obtain a recombinant vector;
4) introducing the recombinant vector in step 3) into host strain to obtain a recombinant E. coli strain;
5) extracting and purifying the recombinant vector from the recombinant E. coli strain after culture of the recombinant E. coli strain in step 4); where the culture is conducted in a medium with ¹⁵NH₄Cl as a sole source of nitrogen and/or ¹³C-glucose as a sole source of carbon;
6) digesting the recombinant vector obtained in step 5) using the first restriction endonuclease and the second restriction endonuclease to obtain an asymmetric double-stranded DNA (dsDNA) structure, and isolating two ssDNAs of unequal lengths, with one of the two ssDNAs as a target sequence; and
7) recovering the target sequence in the two ssDNAs in step 6) to obtain the stable isotope-labeled target ssDNA.

2. The method according to claim 1, wherein in step 1), the first restriction endonuclease is KpnI and the second restriction endonuclease is BamHI; or, the first restriction endonuclease is KpnI-HF and the second restriction endonuclease is BamHI-HF; or, the first restriction endonuclease is Pst I-HF and the second restriction endonuclease is Hind III-HF; or, the first restriction endonuclease is Kpn I-HF and the second restriction endonuclease is Hind III-HF.

3. The method according to claim 1, wherein in step 6), the isolating is isolating by urea-polyacrylamide gel electrophoresis.

4. The method according to claim 1, wherein a short one of the two ssDNAs of unequal lengths in step 6) is a target sequence.

5. The method according to claim 4, wherein the isolating is isolating by urea-polyacrylamide gel electrophoresis.

6. The method according to claim 1, wherein in step 2), the ligating is conducted by ligating every 3 to 4 repeating units in tandem as a large repeating unit, and then ligating the large repeating unit in tandem by a linker sequence.

7. The method according to claim 1, wherein, in step 2), the fusion sequence has a length of not more than 2,000 nt.

8. The method according to claim 7, wherein in step 2), the ligating is conducted by ligating every 3 to 4 repeating units in tandem as a large repeating unit, and then ligating the large repeating unit in tandem by a linker sequence.

9. The method according to claim 1, wherein in step 3), the high-copy vector comprises pUC57.

10. The method according to claim 1, wherein in step 1), the target sequence comprises telomere ssDNA, human c-Myc promoter ssDNA, or human immunodeficiency virus (HIV) ssDNA; the telomere ssDNA has the nucleotide sequence set forth in SEQ ID NO: 1; the human c-Myc promoter ssDNA has the nucleotide sequence set forth in SEQ ID NO: 2; and the HIV ssDNA has the nucleotide sequence set forth in SEQ ID NO: 3.

11. The method according to claim 10, wherein when the target sequence is telomere ssDNA, the ligating in step 2) is conducted by ligating 12 repeating units in tandem to obtain a fusion sequence;
when the target sequence is human c-Myc promoter ssDNA, the ligating is conducted by ligating 20 repeating units in tandem to obtain the fusion sequence; and
when the target sequence is HIV ssDNA, the ligating is conducted by ligating 15 repeating units in tandem to obtain the fusion sequence.

12. The method according to claim 1, wherein the ¹⁵NH₄Cl in the medium in step 5) has a concentration of at least 1 g/L; the ¹³C-glucose in the medium has a concentration of at least 4 g/L.

13. The method according to claim 1, wherein step 7) further includes recovering another ssDNA in addition to the target sequence.

14. A method for preparing a product for screening a medicament targeting HIV long terminal repeat (LTR)-III G-quadruplex (G4), comprising using a stable isotope-labeled ssDNA obtained by the method according to claim 1.

15. The method according to claim 14, wherein in step 1), the first restriction endonuclease is KpnI and the second restriction endonuclease is BamHI; or, the first restriction endonuclease is KpnI-HF and the second restriction endonuclease is BamHI-HF; or, the first restriction endonuclease is Pst I-HF and the second restriction endonuclease is Hind III-HF; or, the first restriction endonuclease is Kpn I-HF and the second restriction endonuclease is Hind III-HF.

16. The method according to claim 14, wherein in step 6), the isolating is isolating by urea-polyacrylamide gel electrophoresis.

17. The method according to claim 14, wherein a short one of the two ssDNAs of unequal lengths in step 6) is a target sequence.

18. The method according to claim 14, wherein in step 2), the ligating is conducted by ligating every 3 to 4 repeating units in tandem as a large repeating unit, and then ligating the large repeating unit in tandem by a linker sequence.

19. The method according to claim 14, wherein, in step 2), the fusion sequence has a length of not more than 2,000 nt.

20. The method according to claim 14, wherein in step 3), the high-copy vector comprises pUC57.
